SEQUENCE LISTING (1 sequence)

SEQ ID NO: 1 - protein, 9 aa, homo sapiens
KVPRNQDWL

(19) United States
(12) Patent Application Publication
(10) Pub. No.: US 2017/0216355 A1
(43) Pub. Date: Aug. 3, 2017

(54) Methods and Compositions for Enhancing T Cell Function and Survival

(71) Applicant: Augusta University Research Institute, Inc., Augusta, GA (US)

(72) Inventors: Samir N. Khleif, Silver Spring, MD (US); Mikayel Mkrtichyan, Millbrae, CA (US)

(21) Appl. No.: 15/422,706

(22) Filed: Feb. 2, 2017

Related U.S. Application Data
(60) Provisional application No. 62/290,227, filed on Feb. 2, 2016.

(51) Int. Cl. A61K 35/17; A61K 31/427; A61K 31/5377; A61K 39/00; A61K 31/52

(52) U.S. Cl. CPC A61K 35/17; A61K 39/0011; A61K 31/52; A61K 31/5377; A61K 31/427; A61K 2035/122

(57) ABSTRACT

It has been discovered that the inhibition of PI3K-δ, but not PI3K-α or PI3K-β, delays the terminal differentiation of CD8 T cell and maintains the T_{CM} phenotype thus enhancing their proliferative ability and survival while maintaining their cytokine and Granzyme B production ability. Methods and compositions for delaying or inhibiting terminal differentiation of CD8 T cells are provided.

### CROSS-REFERENCE TO RELATED APPLICATIONS

Application No. 62/290,227 filed on Feb. 2, 2016, and is incorporated by reference in its entirety.

### FIELD OF THE INVENTION

The invention is generally directed to compositions and methods for modulating T cells.

### REFERENCE TO A “SEQUENCE LISTING”

The Sequence Listing submitted Feb. 1, 2017, as a text file named “2016_18.STtxt” created on Feb. 1, 2017, and having a size of 1 Kb is hereby incorporated by reference pursuant to 37 C.F.R. §1.52(e)(5).

### BACKGROUND OF THE INVENTION

CD8 T cell response comprises effector and memory T cells. There are different subsets of CD8 memory T cells including effector (T_{EM}) and central (T_{CM}) memory T cells. T_{CM} possess an enhanced proliferative ability, greater functionality and a better longevity. Therefore, T_{CM} have superior qualities enabling them to better fight microbial challenges and mediate a therapeutic antitumor immunity when compared to T_{EM} cells.

The differentiation of different T cell subsets is under the control of the PI3K/Akt pathway. In fact, Akt activation was found to regulate the effector/memory CD8 T cell differentiation. It was recently reported that the inhibition of the specific Akt isoforms, Akt1 and Akt2, delays the terminal differentiation of CD8 T cells while enhancing the T_{CM} phenotype. Targeting these specific Akt isoforms therefore enhanced the proliferative ability, longevity and cytokine production in CD8 T cells

It is an object of the invention to provide compositions and methods to inhibit CD8 T cell terminal differentiation.

It is another object of the invention to provide compositions and methods for enhancing an immune response.

### SUMMARY OF THE INVENTION

It has been discovered that the inhibition of PI3K-δ, but not PI3K-α or PI3K-β, delays the terminal differentiation of CD8 T cell and maintains the T_{CM} phenotype thus enhancing their proliferative ability and survival while maintaining their cytokine and Granzyme B production ability. Interestingly, it has been reported the effectiveness of PI3K-δ inhibition in selectively targeting the immunosuppressive regulatory CD4 T cells (Tregs).

The data provided herein show that the functional ability of CD8 T cells treated with a PI3K-δ inhibitor in vivo, where the adoptive cell transfer (ACT) of these cells, in combination with a tumor vaccine, significantly enhanced animal survival and anti-tumor therapeutic ability when compared to ACT of non-treated CD8 T cells.

In cancer immunotherapy, there is a sparsity of drugs that delay the exhaustion of CD8 T cells without impacting their proliferative ability. PI3K-δ is responsible for driving the terminal differentiation of CD8 T cells, and its inhibition delays their exhaustion and enhances their proliferation, cytokine production and subsequently their anti-tumor therapeutic ability.

One embodiment provides a method for delaying or inhibiting terminal differentiation of CD8 T cells in a subject by administering to the subject an effective amount of an inhibitor of PI3K-δ.

Another embodiment provides a method for maintaining T_{CM} phenotype of T cells by contacting the T cells with an effective amount of an inhibitor of PI3K-δ to delay terminal differentiation and maintain the T_{CM} phenotype of the T cells.

Still another embodiment provides a method for inhibiting or delaying exhaustion of CD8 T cells in a subject by administering to the subject an effective amount of an inhibitor of PI3K-δ to enhance the proliferative ability and survival of the CD8 T cells.

Another embodiment provides a method of modulating an immune response by administering an effective amount of an inhibitor of PI3K-δ to enhance the proliferative ability and survival of the CD8 T cells.

Another embodiment provides a method for reducing tumor burden in a subject in need there of by administering to the subject an effective amount of CD8 T cells treated with an inhibitor of PI3K-δ in combination or alternation with a tumor vaccine.

Another embodiment provides a method for treating cancer by administering to a subject in need thereof and effective amount of an inhibitor of PI3K-δ to delay or inhibit CD8 T cell exhaustion and enhance CD8 T cell proliferation and CD8 T cell cytokine production.

Another embodiment provides an immunotherapy composition comprising CD8 T cells treated with an effective amount of an inhibitor of PI3K-δ to delay or inhibit CD8 T cell exhaustion and enhance CD8 T cell proliferation and CD8 T cell cytokine production.

Another embodiment provides an immunotherapy regimen by administering to a subject in need thereof CD8 T cells treated with an effective amount of an inhibitor of PI3K-δ to delay or inhibit CD8 T cell exhaustion and enhance CD8 T cell proliferation and CD8 T cell cytokine production.

### BRIEF DESCRIPTION OF THE DRAWINGS

FIGS. 1A-1P are scatter plots of CD8 T cells from pMel-1 mice stimulated with gp100₂₅₋₃₃ peptide at a 1 μM concentration. On Days 7, 14 and 21, cells were re-stimulated with gp100₂₅₋₃₃ peptide at a 1 μM concentration using feeder cells (irradiated WT splenocytes, 4000 Rads) at 1:1 ratio using the same culture conditions. Cells were stained with the following surface marker antibodies (BD Biosciences): APC-Cy7 labeled anti-CD8, FITC labeled anti-Vβ13, PE labeled anti-CD62L, APC labeled anti-CD44, PE-CF594 labeled anti-CD127, APC labeled anti-KLRG-1 in addition to the viability stain 7AAD. All the analyses were performed on viable (7AAD−), Vβ13+CD8+ T cells. FIGS. 1A-1D are cells stimulated on day three and treated with no inhibitor, GDC 11 nM, GDC 33 nM, and GDC 99 nM, respectively. FIGS. 1E-1H are cells stimulated on day seven and treated with no inhibitor, GDC 11 nM, GDC 33 nM, and GDC 99 nM, respectively. FIGS. 1I-1L are cells stimulated twice and harvested on day seven and treated with no inhibitor, GDC 11 nM, GDC 33 nM, and GDC 99 nM, respectively. FIGS. 1M-1P are cells stimulated three times and harvested on day seven and treated with no inhibitor, GDC 11 nM, GDC 33 nM, and GDC 99 nM, respectively.

FIG. 2A is a line graph of T cells treated with GDC at 239 nM, 99 nM, 33 nM, and no inhibitor. FIG. 2B is a bar graph of Fold Expansion on cells stimulated once and harvested on day seven that were treated (from left to right) with GP100 GDC 11 nM, GDC 33 nM, and GDC 99 nM. FIG. 2C is a bar graph of Fold Expansion on cells stimulated twice and harvested on day seven that were treated (from left to right) with GP100 GDC 11 nM, GDC 33 nM, and GDC 99 nM. FIG. 2D is a bar graph of Fold Expansion on cells stimulated three times and harvested on day seven that were treated (from left to right) with GP100 GDC 11 nM, GDC 33 nM, and GDC 99 nM.

FIGS. 3A-3D are line graphs of percentage of the max versus CD62L of cells treated with GDC at 99 nM, 33 nM, 11 nM, or no inhibitor. FIG. 3A shows cells harvested on day 3 after one stimulation. FIG. 3B shows cells harvested on day 7 after one stimulation. FIG. 3C shows cells harvested on day 7 after two stimulations. FIG. 3D shows cells harvested on day 7 after three stimulations.

FIG. 4 is a bar graph of IL-2 (normalized) for cells treated with GP100 1 μM or GDC 99 nM after two stimulations or three stimulations.

FIG. 5A is a bar graph of IFN-γ (normalized) for cells treated with GP100 1 μM or GDC 99 nM after two stimulations or three stimulations. FIG. 5B is a bar graph of TNF (normalized) for cells treated with GP100 1 μM or GDC 99 nM after two stimulations or three stimulations.

FIG. 6A is a bar graph of IFN-γ (normalized) for cells treated with GP100 1 μM or A66 288 nM after two stimulations or three stimulations. FIG. 6B is a bar graph of TNF (normalized) for cells treated with GP100 1 μM or A66 288 nM after two stimulations or three stimulations. FIG. 6C is a bar graph of IFN-γ (normalized) for cells treated with GP100 1 μM or TGX 45 nM after two stimulations or three stimulations. FIG. 6D is a bar graph of TNF (normalized) for cells treated with GP100 1 μM or TGX 45 nM after two stimulations or three stimulations. FIG. 6E is a bar graph of IFN-γ (normalized) for cells treated with GP100 1 μM or CAL-100 202.5 nM after two stimulations or three stimulations. FIG. 6F is a bar graph of TNF (normalized) for cells treated with GP100 1 μM or CAL-101 after two stimulations or three stimulations.

FIGS. 7A-7F are scatter plots of granzyme B versus CD8 of cells harvested on day seven and stimulated once. FIG. 7A shows naïve cells. FIG. 7B shows cells with no treatment. FIG. 7C shows cells treated with GDC 99 nM. FIG. 7D shows cells treated with A66 288 nM. FIG. 7E shows cells treated with TGX 45 nM. FIG. 7F shows cells treated with CAL-101 22.5 nM. FIGS. 7G-7L are scatter plots of granzyme B versus CD8 of cells harvested on day seven and stimulated twice. FIG. 7G shows naïve cells. FIG. 7H shows cells with no treatment. FIG. 7I shows cells treated with GDC 99 nM. FIG. 7J shows cells treated with A66 288 nM. FIG. 7K shows cells treated with TGX 45 nM. FIG. 7L shows cells treated with CAL-101 22.5 nM.

FIGS. 8A-8D are scatter plots of CD62L versus CD44 for cells harvested on day 3 with one stimulation. FIGS. 8E-8H are scatter plots of CD62L versus CD44 for cells harvested on day 7 with one stimulation. FIGS. 8I-8L are scatter plots of CD62L versus CD44 for cells harvested on day 7 with two stimulations. FIGS. 8M-8P are scatter plots of CD62L versus CD44 for cells harvested on day 7 with three stimulations.

FIG. 9A is a graph of percentage of the max versus VCT for cells treated with A66 288 nM, A66 96 nM, A66 32 nM, or no inhibitor. FIG. 9B is a graph of percentage of the max versus VCT for cells treated with TGX 45 nM, TGX 15 nM, TGX 5 nM, or no inhibitor. FIG. 9C is a graph of percentage of the max versus VCT for cells treated with CAL-101 22.5 nM, CAL-101 7.5 nM, CAL-101 2.5 nM, or no inhibitor.

FIG. 10A is a bar graph of Fold Expansion for cells harvested on day 7 with one stimulation treated with 1 μM GP100, 32 nM A66, 96 nM A66, or 288 nM A66. FIG. 10B is a bar graph of Fold Expansion for cells harvested on day 7 with two stimulations treated with 1 μM GP100, 32 nM A66, 96 nM A66, or 288 nM A66. FIG. 10C is a bar graph of Fold Expansion for cells harvested on day 7 with three stimulations treated with 1 μM GP100, 32 nM A66, 96 nM A66, or 288 nM A66. FIG. 10D is a bar graph of Fold Expansion for cells harvested on day 7 with one stimulation and treated with 1 μM GP100, 5 nM TGX, 15 nM TGX, or 45 nM TGX. FIG. 10E is a bar graph of Fold Expansion for cells harvested on day 7 with two stimulations treated with 1 μM GP100, 5 nM TGX, 15 nM TGX, or 45 nM TGX. FIG. 10F is a bar graph of Fold Expansion for cells harvested on day 7 with three stimulations treated with 1 μM GP100, 5 nM TGX, 15 nM TGX, or 45 nM TGX. FIG. 10G is a bar graph of Fold Expansion for cells harvested on day 7 with one stimulation for cells treated with 1 μM GP100, 0.28 nM CAL-101, 0.83 nM CAL-101, 2.5 nM CAL-101, 7.5 nM CAL-101, 22.5 CAL-101, 67.5 nM CAL-101, and 202.5 nM CAL-101. FIG. 10H is a bar graph of Fold Expansion for cells harvested on day 7 with two stimulations for cells treated with 1 μM GP100, 0.28 nM CAL-101, 0.83 nM CAL-101, 2.5 nM CAL-101, 7.5 nM CAL-101, 22.5 CAL-101, 67.5 nM CAL-101, and 202.5 nM CAL-101. FIG. 10I is a bar graph of Fold Expansion for cells harvested on day 7 with three stimulations for cells treated with 1 μM GP100, 0.28 nM CAL-101, 0.83 nM CAL-101, 2.5 nM CAL-101, 7.5 nM CAL-101, 22.5 CAL-101, 67.5 nM CAL-101, and 202.5 nM CAL-101.

FIG. 11A is graph of percentage of the max versus CD62L for cells harvested on day 3 after one stimulation and treated with 288 nM A66, 96 nM A66, 32 nM A66, or no inhibitor. FIG. 11B is graph of percentage of the max versus CD62L for cells harvested on day 7 after one stimulation and treated with 288 nM A66, 96 nM A66, 32 nM A66, or no inhibitor. FIG. 11C is graph of percentage of the max versus CD62L for cells harvested on day 7 after two stimulations and treated with 288 nM A66, 96 nM A66, 32 nM A66, or no inhibitor. FIG. 11D is graph of percentage of the max versus CD62L for cells harvested on day 7 after three stimulations and treated with 288 nM A66, 96 nM A66, 32 nM A66, or no inhibitor. FIG. 11E is graph of percentage of the max versus CD62L for cells harvested on day 3 after one stimulation and treated with 45 nM TGX, 15 nM TGX, 5 nM TGX, or no inhibitor. FIG. 11F is graph of percentage of the max versus CD62L for cells harvested on day 7 after one stimulation and treated with 45 nM TGX, 15 nM TGX, 5 nM TGX, or no inhibitor. FIG. 11G is graph of percentage of the max versus CD62L for cells harvested on day 7 after two stimulations and treated with 45 nM TGX, 15 nM TGX, 5 nM TGX, or no inhibitor. FIG. 11H is graph of percentage of the max versus CD62L for cells harvested on day 7 after three stimulations and treated with 45 nM TGX, 15 nM TGX, 5 nM TGX, or no inhibitor. FIG. 11I is graph of percentage of the max versus CD62L for cells harvested on day 3 after one stimulation and treated with 202.5 nM CAL-101, 67.5 nM CAL-101, 22.5 nM CAL-101, 7.5 nM CAL-101, 2.5 nM CAL-101, 0.83 nM CAL-101, 0.28 nM CAL-101 or no inhibitor. FIG. 11I is graph of percentage of the max versus CD62L for cells harvested on day 3 after one stimulation and treated with 202.5 nM CAL-101, 67.5 nM CAL-101, 22.5 nM CAL-101, 7.5 nM CAL-101, 2.5 nM CAL-101, 0.83 nM CAL-101, 0.28 nM CAL-101 or no inhibitor. FIG. 11J is graph of percentage of the max versus CD62L for cells harvested on day 7 after one stimulation and treated with 202.5 nM CAL-101, 67.5 nM CAL-101, 22.5 nM CAL-101, 7.5 nM CAL-101, 2.5 nM CAL-101, 0.83 nM CAL-101, 0.28 nM CAL-101 or no inhibitor. FIG. 11K is graph of percentage of the max versus CD62L for cells harvested on day 7 after two stimulations and treated with 202.5 nM CAL-101, 67.5 nM CAL-101, 22.5 nM CAL-101, 7.5 nM CAL-101, 2.5 nM CAL-101, 0.83 nM CAL-101, 0.28 nM CAL-101 or no inhibitor. FIG. 11L is graph of percentage of the max versus CD62L for cells harvested on day 7 after three stimulations and treated with 202.5 nM CAL-101, 67.5 nM CAL-101, 22.5 nM CAL-101, 7.5 nM CAL-101, 2.5 nM CAL-101, 0.83 nM CAL-101, 0.28 nM CAL-101 or no inhibitor.

FIG. 12A is a bar graph of IL-2 (normalized) for cells treated with 1 μM GP100 or 288 nM A66 that have been stimulated twice or three times. FIG. 12B is a bar graph of IL-2 (normalized) for cells treated with 1 GP100 or 45 nM TGX that have been stimulated twice or three times. FIG. 12C is a bar graph of IL-2 (normalized) for cells treated with 1 μM GP100 or 202.5 nM CAL-100 that have been stimulated twice or three times.

FIG. 13 is a diagram of an treatment regimen.

FIG. 14A is a line graph of tumor volume (cm³) versus days post tumor inoculation in non-treated mice. FIG. 14B is a line graph of tumor volume (cm³) versus days post tumor inoculation in mice treated with CyFlu. FIG. 14C is a line graph of tumor volume (cm³) versus days post tumor inoculation in mice treated with CD8 cells. FIG. 14D is a line graph of tumor volume (cm³) versus days post tumor inoculation in mice treated with CD8/CAL-CD8 T cells treated with CAL-101. FIG. 14E is a line graph of tumor volume (cm³) versus days post tumor inoculation in mice treated with a vaccine. FIG. 14F is a line graph of tumor volume (cm³) versus days post tumor inoculation in mice treated with CyFlu and vaccine. FIG. 14G is a line graph of tumor volume (cm³) versus days post tumor inoculation in mice treated with CD8 cells and vaccine. FIG. 14H is a line graph of tumor volume (cm3) versus days post tumor inoculation in mice treated with CD8/CAL-CD8 T cells and vaccine.

FIG. 15 is a Kaplan-Meier graph.

FIG. 16A is a scatter plot of CD62L versus CD44 for untreated cells harvested at day 3 and stimulated once. FIG. 16B is a scatter plot of CD62L versus CD44 for cells harvested at day 3, stimulated once, and treated with 32 nM A66. FIG. 16C is a scatter plot of CD62L versus CD44 for cells harvested at day 3, stimulated once, and treated with 96 nM A66. FIG. 16D is a scatter plot of CD62L versus CD44 for cells harvested at day 3, stimulated once, and treated with 288 nM A66. FIG. 16E is a scatter plot of CD62L versus CD44 for untreated cells harvested at day 7 and stimulated once. FIG. 16F is a scatter plot of CD62L versus CD44 for cells harvested at day 7, stimulated once, and treated with 32 nM A66. FIG. 16G is a scatter plot of CD62L versus CD44 for cells harvested at day 7, stimulated once, and treated with 96 nM A66. FIG. 16H is a scatter plot of CD62L versus CD44 for cells harvested at day 7, stimulated once, and treated with 288 nM A66. FIG. 16I is a scatter plot of CD62L versus CD44 for untreated cells harvested at day 3 and stimulated twice. FIG. 16J is a scatter plot of CD62L versus CD44 for cells harvested at day 7, stimulated twice, and treated with 32 nM A66. FIG. 16K is a scatter plot of CD62L versus CD44 for cells harvested at day 7, stimulated twice, and treated with 96 nM A66. FIG. 16L is a scatter plot of CD62L versus CD44 for cells harvested at day 7, stimulated twice, and treated with 288 nM A66. FIG. 16M is a scatter plot of CD62L versus CD44 for untreated cells harvested at day 3 and stimulated three times. FIG. 16J is a scatter plot of CD62L versus CD44 for cells harvested at day 7, stimulated three times, and treated with 32 nM A66. FIG. 16O is a scatter plot of CD62L versus CD44 for cells harvested at day 7, stimulated three times, and treated with 96 nM A66. FIG. 16P is a scatter plot of CD62L versus CD44 for cells harvested at day 7, stimulated three times, and treated with 288 nM A66.

FIG. 17A is a scatter plot of CD62L versus CD44 for untreated cells harvested on day 3 after one stimulation. FIGS. 17B-17D are scatter plots of CD62L versus CD44 for cells harvested on day 3 after one stimulation and treated with 5 nM TGX, 15 nM TGX, or 45 nM TGX, respectively. FIGS. 17E-17G are scatter plots of CD62L versus CD44 for cells harvested on day 7 after one stimulation with either no treatment or treated with 5 nM TGX, 15 nM TGX, or 45 nM TGX, respectively. FIGS. 17I-17L are scatter plots of CD62L versus CD44 for cells harvested on day 7 after two stimulations with either no treatment or treated with 5 nM TGX, 15 nM TGX, or 45 nM TGX, respectively. FIGS. 17M-17P are scatter plots of CD62L versus CD44 for cells harvested on day 7 after three stimulations with either no treatment or treated with 5 nM TGX, 15 nM TGX, or 45 nM TGX, respectively.

FIGS. 18A-18H are scatter plots of CD62L versus CD44 for cells harvested on day 3 after one stimulation with either no treatment or treated with 0.28 nM CAL-101, 0.83 nM CAL-101, 2.5 nM CAL-101, 7.5 nM CAL-101, 22.5 nM CAL-101, 67.5 nM CAL-101, or 202.5 nM CAL-101, respectively. FIGS. 18I-18P are scatter plots of CD62L versus CD44 for cells harvested on day 7 after one stimulation with either no treatment or treated with 0.28 nM CAL-101, 0.83 nM CAL-101, 2.5 nM CAL-101, 7.5 nM CAL-101, 22.5 nM CAL-101, 67.5 nM CAL-101, or 202.5 nM CAL-101, respectively. FIGS. 18Q-18X are scatter plots of CD62L versus CD44 for cells harvested on day 7 after two stimulations with either no treatment or treated with 0.28 nM CAL-101, 0.83 nM CAL-101, 2.5 nM CAL-101, 7.5 nM CAL-101, 22.5 nM CAL-101, 67.5 nM CAL-101, or 202.5 nM CAL-101, respectively. FIGS. 18Y-18FF are scatter plots of CD62L versus CD44 for cells harvested on day 7 after three stimulations with either no treatment or treated with 0.28 nM CAL-101, 0.83 nM CAL-101, 2.5 nM CAL-101, 7.5 nM CAL-101, 22.5 nM CAL-101, 67.5 nM CAL-101, or 202.5 nM CAL-101, respectively.

FIG. 19A is a bar graph of IL-2 (normalized) produced by cells treated with 1 μM GP100, 0.28 nM CAL-101, 0.83 nM CAL-101, 2.5 nM CAL-101, 7.5 nM CAL-101, 22.5 nM CAL-101, 67.5 nM CAL-101, or 202.5 nM CAL-101 after two stimulations or three stimulations. FIG. 19A is a bar graph of IFN-γ (normalized) produced by cells treated with 1 μM GP100, 0.28 nM CAL-101, 0.83 nM CAL-101, 2.5 nM CAL-101, 7.5 nM CAL-101, 22.5 nM CAL-101, 67.5 nM CAL-101, or 202.5 nM CAL-101 after two stimulations or three stimulations. FIG. 19C is a bar graph of TNF (normalized) produced by cells treated with 1 μM GP100, 0.28 nM CAL-101, 0.83 nM CAL-101, 2.5 nM CAL-101, 7.5 nM CAL-101, 22.5 nM CAL-101, 67.5 nM CAL-101, or 202.5 nM CAL-101.

FIGS. 20A-20D are scatter plots of CD62L versus CD44 for cells harvested at day 3 after stimulation and are untreated, or treated with 11 nM GDC, 33 nM GDC, 99 nM GDC, respectively. FIGS. 20E-20H are scatter plots of CD62L versus CD44 for cells harvested at day 7 after stimulation and are untreated, or treated with 11 nM GDC, 33 nM GDC, 99 nM GDC, respectively. FIGS. 20I-20L are scatter plots of CD62L versus CD44 for cells harvested at day 7 after two stimulations and are untreated, or treated with 11 nM GDC, 33 nM GDC, 99 nM GDC, respectively. FIGS. 20M-20P are scatter plots of CD62L versus CD44 for cells harvested at day 7 after three stimulations and are untreated, or treated with 11 nM GDC, 33 nM GDC, 99 nM GDC, respectively.

FIG. 21A is graph of percentage of the max versus VCT for cells treated with 279 nM GDC, 99 nM GDC, 33 nM GDC, or untreated, respectively. FIG. 21B is bar graph of fold expression for cells on day 3 after one stimulation that are untreated, treated with 11 nM GDC, 33 nM GDC, or 99 nM GDC, respectively. FIG. 21C is bar graph of fold expression for cells on day 7 after two stimulations that are untreated, treated with 11 nM GDC, 33 nM GDC, or 99 nM GDC, respectively. FIG. 21D is bar graph of fold expression for cells on day 7 after three stimulations that are untreated, treated with 11 nM GDC, 33 nM GDC, or 99 nM GDC, respectively.

FIG. 22A is a graph of percentage of the max versus CD62L for cells on day 3 after stimulation that are treated with 99 nM GDC, 33 nM GDC, 11 nM GDC, or untreated respectively. FIG. 22B is a graph of percentage of the max versus CD62L for cells on day 7 after stimulation that are treated with 99 nM GDC, 33 nM GDC, 11 nM GDC, or untreated respectively. FIG. 22C is a graph of percentage of the max versus CD62L for cells on day 7 after two stimulations that are treated with 99 nM GDC, 33 nM GDC, 11 nM GDC, or untreated respectively. FIG. 22D is a graph of percentage of the max versus CD62L for cells on day 7 after three stimulations that are treated with 99 nM GDC, 33 nM GDC, 11 nM GDC, or untreated respectively.

FIG. 23 is a bar graph of IL-2 (normalized) for untreated cells or cells treated with 99 nM GDC after two stimulations or three stimulations respectively.

FIG. 24A is bar graph of IFN-γ (normalized) for untreated cells or cells treated with 99 nM GDC after two stimulations or three stimulations respectively. FIG. 24B is bar graph of TNF-α (normalized) for untreated cells or cells treated with 99 nM GDC after two stimulations or three stimulations respectively.

FIG. 25A is a bar graph of INF-γ (pg/ml) from untreated cells or cells treated with 288 nM A66 and stimulated twice or three times. FIG. 25B is a bar graph of TNFα (pg/ml) from untreated cells or cells treated with 288 nM A66 and stimulated twice or three times. FIG. 25C is a bar graph of INF-γ (pg/ml) from untreated cells or cells treated with 45 nM TGX and stimulated twice or three times. FIG. 25D is a bar graph of TNFα (pg/ml) from untreated cells or cells treated with 45 nM TGX and stimulated twice or three times. FIG. 25E is a bar graph of INF-γ (pg/ml) from untreated cells or cells treated with 202.5 nM CAL-101 and stimulated twice or three times. FIG. 25F is a bar graph of TNFα (pg/ml) from untreated cells or cells treated with 202.5 nM CAL-101 and stimulated twice or three times.

FIGS. 26A-26D are scatter plots of CD62L versus CD44 for cells harvested at day 3 after stimulation and are untreated, treated with 288 nM A66, 45 nM TGX, or 202.5 nM CAL-100, respectively. FIGS. 26E-26H are scatter plots of CD62L versus CD44 for cells harvested at day 7 after stimulation and are untreated, treated with 288 nM A66, 45 nM TGX, or 202.5 nM CAL-100, respectively. FIGS. 26I-26L are scatter plots of CD62L versus CD44 for cells harvested at day 7 after stimulation twice and are untreated, treated with 288 nM A66, 45 nM TGX, or 202.5 nM CAL-100, respectively. FIGS. 26M-26P are scatter plots of CD62L versus CD44 for cells harvested at day 7 after stimulation three times and are untreated, treated with 288 nM A66, 45 nM TGX, or 202.5 nM CAL-100, respectively.

FIG. 27A is a graph of percentage of the max versus VCT for cells treated with 288 nM AGG, 96 nM A66, 32 nM A66, or no inhibitor respectively. FIG. 27B is a graph of percentage of the max versus VCT for cells treated with 45 nM TGX, 15 nM TGX, 5 nM TGX, or no inhibitor respectively. FIG. 27C is a graph of percentage of the max versus VCT for cells treated with 22.5 nM CAL-101, 7.5 nM CAL-101, 2.5 nM CAL-101, or no inhibitor respectively.

FIG. 28A is a bar graph of fold expansion for cells harvested at day 7 after stimulation and are untreated or treated with 32 nM A66, 96 nM A66, 288 nM A66 respectively. FIG. 28B is a bar graph of fold expansion for cells harvested at day 7 after two stimulations and are untreated or treated with 32 nM A66, 96 nM A66, 288 nM A66 respectively. FIG. 28C is a bar graph of fold expansion for cells harvested at day 7 after three stimulations and are untreated or treated with 32 nM A66, 96 nM A66, 288 nM A66 respectively. FIG. 28D is a bar graph of fold expansion for cells harvested at day 7 after stimulation and are untreated or treated with 5 nM TGX, 15 nM TGX, 45 nM TGX respectively. FIG. 28E is a bar graph of fold expansion for cells harvested at day 7 after two stimulations and are untreated or treated with 5 nM TGX, 15 nM TGX, 45 nM TGX respectively. FIG. 28F is a bar graph of fold expansion for cells harvested at day 7 after three stimulations and are untreated or treated with 5 nM TGX, 15 nM TGX, 45 nM TGX respectively. FIG. 28G is a bar graph of fold expansion for cells harvested at day 7 after stimulation and are untreated or treated with 0.28 nM CAL-101, 0.83 nM CAL-101, 2.5 nM CAL-101, 7.5 nM CAL-101, 22.5 nM CAL-101, 67.5 nM CAL-101, 202.5 nM CAL-101 respectively. FIG. 28H is a bar graph of fold expansion for cells harvested at day 7 after two stimulations and are untreated or treated with 0.28 nM CAL-101, 0.83 nM CAL-101, 2.5 nM CAL-101, 7.5 nM CAL-101, 22.5 nM CAL-101, 67.5 nM CAL-101, 202.5 nM CAL-101 respectively. FIG. 28I is a bar graph of fold expansion for cells harvested at day 7 after three stimulations and are untreated or treated with 0.28 nM CAL-101, 0.83 nM CAL-101, 2.5 nM CAL-101, 7.5 nM CAL-101, 22.5 nM CAL-101, 67.5 nM CAL-101, 202.5 nM CAL-101 respectively.

FIGS. 29A-29F are scatter plots of Granzyme B versus CD8 for cells harvested at day 7 after stimulation and are naïve cells, untreated cells, treated with GDC 99 nM, 288 nM A66, 45 nM TGX, or 22.5 nM CAL-101, respectively. FIGS. 29G-29L are scatter plots of Granzyme B versus CD8 for cells harvested at day 7 after two stimulations and are naïve cells, untreated cells, treated with GDC 99 nM, 288 nM A66, 45 nM TGX, or 22.5 nM CAL-101, respectively.

FIG. 30A is a scatter plot CD62L versus CD44 of cells treated with 1 μM GP100. FIG. 30B is a scatter plot CD62L versus CD44 of cells treated with 1 μM GP100 and scRNA. FIG. 30C is a scatter plot CD62L versus CD44 of cells treated with 1 μM GP100 and siRNA for PI3Kα. FIG. 30D is a scatter plot CD62L versus CD44 of cells treated with 1 μM GP100 and siRNA for PI3Kβ. FIG. 30E is a scatter plot CD62L versus CD44 of cells treated with GP100 and siRNA for PI3Kδ.

FIGS. 31A-31D are graphs of percentage of the max versus CD62L for cells treated with 288 nM A66, 96 nM A66, 32 nM A66, or untreated cells harvested at day 3 with one stimulation, harvested at day 7 with one stimulation, harvested at day 7 with two stimulations, and harvested at day 7 with three stimulations, respectively. FIGS. 31E-31F are graphs of percentage of the max versus CD62L for cells treated with 45 nM TGX, 15 nM TGX, 5 nM TGX, or untreated cells harvested at day 3 with one stimulation, harvested at day 7 with one stimulation, harvested at day 7 with two stimulations, and harvested at day 7 with three stimulations, respectively. FIGS. 31I-31L are graphs of percentage of the max versus CD62L for cells treated with 202.5 nM CAL-101, 67.5 nM CAL-101, 22.5 nM CAL-101, 7.5 nM CAL-101, 2.5 nM CAL-101, 0.83 nM CAL-101, 0.28 nM CAL-101 or untreated cells harvested at day 3 with one stimulation, harvested at day 7 with one stimulation, harvested at day 7 with two stimulations, and harvested at day 7 with three stimulations, respectively.

FIG. 32A is bar graph of IL-2 (normalized) from untreated cells and cells treated with 288 nM A66 after two or three stimulations. FIG. 32B is bar graph of IL-2 (normalized) from untreated cells and cells treated with 45 nM TGX after two or three stimulations. FIG. 32C is bar graph of IL-2 (normalized) from untreated cells and cells treated with 202.5 nM CAL-101 after two or three stimulations.

FIG. 33 is a bar graph of pAkt/Akt protein levels (normalized) for (from left to right) untreated cells, cells treated with 202.5 nM CAL-101, cells treated with 45 nM TGX, cells treated with 288 nM A66 for pAkt1/Akt1, pAkt2/Akt2 and pAkt3/Ak3, respectively.

### DETAILED DESCRIPTION OF THE INVENTION

### I. Definitions

The use of the terms “a,” “an,” “the,” and similar referents in the context of describing the presently claimed invention (especially in the context of the claims) are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx. +/−10%; in other embodiments the values may range in value either above or below the stated value in a range of approx. +/−5%; in other embodiments the values may range in value either above or below the stated value in a range of approx. +/−2%; in other embodiments the values may range in value either above or below the stated value in a range of approx. +/−1%. The preceding ranges are intended to be made clear by context, and no further limitation is implied. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

### II. Methods for Modulating Immune Responses

In response to antigen encounter, CD8 T cell response comprises effector and memory T cells. CD8 memory T cells include several subtypes including T_{CM} and T_{EM} cells. Memory cells represent earlier stages of differentiation and are superior in their cytotoxic ability against microbial challenges and mediation of therapeutic antitumor immunity when compared to effector cells which are terminally differentiated. Out of the two memory subtypes, T_{CM} cells are by far superior to T_{EM} cells, due to their greater proliferative capacity upon antigen re-encounter.

The PI3K/Akt pathway governs many T cell functions, including proliferation, survival, migration, and metabolism. Specifically, the differentiation of CD8 cells into memory T cells is coordinated by PI3K/Akt signaling. Continuous activation of this pathway drives the terminal differentiation while its inhibition (at the level of Akt or the downstream mTOR) enhances the quality of CD8 T cells by prompting a memory phenotype.

It was recently reported that the specific Akt1 and Akt2 isoforms are the drivers of terminal differentiation of CD8 T cells and that their inhibition preserves a healthy reservoir of highly proliferative and functionally superior memory CD8 T cells.

Here, it is shown, for the first time, that the PI3K-δ isoform drives the terminal differentiation of CD8 T cells and that its inhibition enhances their survival and proliferative ability upon re-encountering the antigen by preserving a remarkably high percentage of memory CD8 T cells.

We report that the inhibition of PI3K-δ, but not PI3K-α or PI3K-β, enhances the proliferative ability of CD8 cells and maintains a high CD62L expression level and IL-2 secretion.

The data in the Examples show that the inhibition of PI3K-δ in CD8 T cells greatly enhances the anti-tumor therapeutic ability of these cells when adoptively transferred into tumor bearing mice.

Unlike the inhibition of Akt1 and Akt2 that resulted in a high expression level of CD127 and a low level of KLRG-1, the inhibition of PI3K-δ did not have a significant effect on the expression levels of these markers (data not shown). This could be attributed to the involvement of the upstream PI3K in many different pathways, where compensatory mechanisms could overcome its inhibition.

We report that PI3K-δ inhibition enhances the proliferation and therefore the number of available cytotoxic CD8 T cells. This is in contrast to the effect of mTOR inhibition that has been shown to modulate the functionality and phenotype of CD8 T cells, while simultaneously decreasing their proliferation in the tumor micro-environment. Not only does mTOR inhibition directly mitigate CD8 T cells, it also significantly increases the numbers of the suppressive regulatory T cell (Treg). mTOR inhibition by rapamycin supports the proliferation and survival of Treg cells due to a feedback loop where mTOR inhibition results in PI3K-dependent Akt activation. Therefore, antibody-based depletion of Treg cells was proposed to overcome CD8 T cell inhibition when mTOR inhibition is used to augment memory T cells. However, this strategy does not negate the inhibitory effect exerted directly on CD8 T cell proliferation. Conversely, we demonstrate that inhibition of the PI3K/Akt pathway at the level of Akt, and PI3K significantly enhances the ability of CD8 T cells to proliferate. Additionally, it has been demonstrated that PI3K/Akt pathway inhibitors selectively target and decrease Tregs within the tumor micro-environment²⁵. Specifically, PI3K-δ isoform has been recently reported to effectively mitigate Tregs thus enhancing anti-tumor immunity. Therefore, using PI3K-δ inhibitors can enhance the effector arm by enhancing memory CD8 T cells and inhibit the suppressive arm by selectively inhibiting Treg cells.

As CD8 T cells differentiate from naïve to effector cells, they lose their ability to produce IL-2. Here, we show that PI3K inhibition in CD8 T cells, in particular the inhibition of the PI3K-δ isoform maintains a higher level of IL-2 secretion. Furthermore, we demonstrate that the inhibition of the PI3K-δ isoform does not affect the CD8 T cells' ability to secrete TNF, IFNγ and Granzyme B. This suggests that PI3K-δ inhibition enhances the proliferative ability and longevity of CD8 T cells without affecting their cytotoxic functionality.

It has been reported that ACT of tumor-reactive CD8 T_{CM} cells is superior in mediating therapeutic antitumor immunity, due to their greater proliferative ability upon antigen re-encounter. Here we demonstrate that treating tumor specific CD8 T cells with a PI3K-δ inhibitor greatly enhances their anti-tumor ability when adoptively transferred into tumor bearing mice. This is not surprising given the enhanced proliferation, survival and functionality of inhibitor treated cells in vitro. Furthermore, the therapeutic ability of these cells was greatly enhanced with the combination of a tumor specific vaccine. Memory CD8 T cells respond to this re-encounter with the antigen (vaccine) by proliferating more robustly than exhausted effector CD8 T cells. Thus, the preservation of memory cells using only PI3K-δ inhibition produces an enhanced cytotoxic anti-tumor ability. This was demonstrated by a remarkable slowdown of tumor growth and a significant enhancement of animal survival.

These findings define a new and vital role for the PI3K-δ isoform in T cell biology. The data demonstrate that PI3K-δ is the isoform responsible for driving the terminal differentiation of CD8 T cells and that targeting this isoform can modulate the differentiation of effector and memory CD8 T cells. This adds to the significant roles that PI3K-δ has in different T cells subsets. In addition to its definition as a key controller of the suppressive Tregs, it has been recently reported that PI3K-δ controls, at least in part, the magnitude of CD8 response to the intracellular infection with L. monocytogenes and the ability to produce long-lived memory T cells. This has important clinical implications for the use of PI3K-δ inhibitors to modulate both Tregs and CD8 T cells.

In summary, the data show that PI3K-δ, but not PI3K-α or PI3K-β, drives the terminal differentiation of CD8 T cells and that its inhibition enhances the memory phenotype, improves CD8 T cell survival and enhances their proliferative potential while maintaining their ability to produce cytotoxic cytokines and Granzyme B. These findings translate into anti-tumor therapeutic efficacy where the ACT of PI3K-δ treated CD8 T cells in an animal tumor model greatly slows down tumor growth and prolongs animal survival.

Agents with the ability to delay terminal differentiation of CD8 T cells without affecting their effector function and proliferation are needed. A strategy that enhances the memory phenotype, proliferative potential and survival without affecting the effector function of CD8 T cells by targeting PI3K-δ is provided. The data have significant clinical implications and strongly suggest the clinical use of PI3K-δ inhibitors as potent modulators of the immune response as part of different cancer immune therapy strategies.

### EXAMPLES

### Materials and Methods

### Mice and Reagents

pMel-1 mice (B6.Cg-Thy1^{a}/Cy Tg(TcraTcrb)8Rest/J) were used for in vitro experiments. Briefly, these mice carry a rearranged T cell receptor transgene (Vβ13) specific for the mouse homologue (pmel-17) of human (gp100). For feeder cells, female C57BL/6(H-2b) wild-type (WT) mice were used. For in vivo experiments, 4-6 week old WT female mice were used. (All mice were purchased from the Jackson Laboratory and housed under pathogen-free conditions).

All the inhibitors were purchased from Selleckchem. GDC-0941 is a pan PI3K inhibitor with an IC50 of 3 nM for p110α, 33 nM for p110β, 3 nM for p110δ and 75 nM for p110γ. This inhibitor was used in vitro at 11, 33, 99 and 279 nM concentrations thus ensuring the inhibition of all three Class 1 isoforms. A66 is a selective p110α inhibitor with an IC50 of 32 nM for the p110α, 236 nM for PI4Kβ, 462 nM for C2β and >1.25 μM for p110δ. A66 was used in vitro at 32, 96 and 288 nM concentrations thus ensuring selectivity to the PI3Kα isoform. TGX-221 is a highly selective PI3Kβ inhibitor with an IC50 of 5 μM for p110α, 5 nM for p100β, 0.1 μM for p100δ and >10 μM for p110γ. In in vitro experiments, TGX-221 was used at 5, 15 and 45 nM to ensure selectivity. CAL-101 is a selective PI3Kδ inhibitor with an IC50 of 820 nM for p110α, 565 nM for p110β, 2.5 nM for p110δ and 89 nM for p110γ. This inhibitor was tested in vitro at the following concentrations; 0.28, 0.83, 2.5, 7.5, 22.5, 67.5, and 202.5 nM to maximize the drug's specificity.

The gp100₂₅₋₃₃ 9-mer peptide (KVPRNQDWL)(SEQ ID NO:1) was purchased from ANASPEC and used for in vitro activation of pMel-1 splenocytes at a 1 μM concentration as previously described.

For in vivo experiments, the vaccine was prepared using the same gp100₂₅₋₃₃ peptide and administered at 100 μg per mouse in combination with PADRE at 10 μg per mouse and Quil A at 25 μg per mouse. Lymphodepletetion of mice was achieved using a combination of 250 mg/kg cyclophosphamide (Sigma) and 50 mg/kg fludarabine (Selleck Chemicals).

### In Vitro Activation of CD8 T Cells

CD8 T cells from pMel-1 mice were activated in vitro as previously described⁵. Briefly, homogenized pMel-1 splenocytes were stimulated with gp100₂₅₋₃₃ peptide at a 1 μM concentration (day 0). Cells were cultured in RPMI 1640 (Lonza) supplemented with 10% FBS, penicillin (100 U/mL), streptomycin (100 mg/mL), 0.1% β-mercaptoethanol (Life Technologies, Invitrogen) and IL-2 (100 U/ml) (Peprotech) at 37° C. with 5% CO2. pMel-1 cells were cultured with or without the different PI3K inhibitors. The concentration of the inhibitors was maintained throughout the culture by changing the media every 48-72 hours.

On Days 7, 14 and 21, cells were re-stimulated with gp100₂₅₋₃₃ peptide at a 1 μM concentration using feeder cells (irradiated WT splenocytes, 4000 Rads) at 1:1 ratio using the same culture conditions.

### Proliferation Assay and Phenotyping of CD8 T Cells

Cells were labeled with 5 μM Violet Cell Trace (VCT) proliferation dye (Life Technologies, Invitrogen) prior to their stimulation (day 0) following the manufacturers' instructions. The proliferation of CD8 T cells was assessed via VCT dye dilution (day 3) using an LSRII SORP with HTS Flow Cytometer (BD Biosciences). The data were analyzed using FlowJo 10 (Tree Star). The cultured cells were harvested on days 3, 7, 14 and 21 to assess their phenotype. Cells were stained with the following surface marker antibodies (BD Biosciences): APC-Cy7 labeled anti-CD8, FITC labeled anti-Vβ13, PE labeled anti-CD62L, APC labeled anti-CD44, PE-CF594 labeled anti-CD127, APC labeled anti-KLRG-1 in addition to the viability stain 7AAD. All the analyses were performed on viable (7AAD−), Vβ13+CD8+ T cells.

For intracellular staining, cells were stained with the fixable near infra-red Live/Dead viability stain (Life Technologies, Invitrogen), and fixed, permeabilized and stained with APC labeled anti-CD8, V450 labeled anti-Vβ13, PE labeled anti-CD62L and PE-CF594 labeled anti-CD44 (BD Biosciences) and FITC labeled Granzyme B (Biolegend). The analyses were performed on viable (Live/Dead negative), Vβ13+CD8+ T cells.

### Cytometric Bead Array

Using the stimulation protocol above, CD8 T cells were harvested on day 7 after the first and second stimulation. Viable (trypan blue negative) cells were co-incubated (at 1:1 ratio) with 1 μM gp100₂₅₋₃₃ pulsated irradiated splenocytes (4000 Rads) for 24 hours using the same culture conditions. Supernatants were collected and the level of IL-2, TNF and IFN-γ was assessed using the mouse Th1/Th2/Th17 Cytokine Kit BD™ Cytometric Bead Array (CBA) kit. Cytokine levels were collected using an LSRII SORP with HTS flow cytometer (BD Biosciences), and analyzed using the FCAP Array Software v3.0 (BD Biosciences).

### In Vivo Tumor Treatment

C57BL/6 female mice were implanted with 400,000 B16 cells/mouse subcutaneously (s.c.) in the right flank on day 0 (B16 expresses gp100 antigen). On day 7, mice were lymphodepleted by s.c. injection of a cocktail of Cyclophosphamide and Fludarabine (CyFlu); 250 mg/kg cyclophosphamide and 50 mg/kg fludarabine. On Day 8, gp100 activated CD8 T cells from pMel-1 mice cultured in the presence or absence of CAL-101 (202.5 nM, for 7 days as described above) were adoptively transferred intravenously (i.v) (1 million cells per mouse). The appropriate groups were vaccinated with gp100 peptide vaccine (gp100₂₅₋₃₃ with PADRE and Quil A as described above) on day 8, 15 and 22 (FIG. 5A). The vaccine doses represented stimulations 2, 3 and 4 of the CD8 T cells. Animal survival and tumor growth was monitored and the animals were sacrificed upon tumor ulceration or reaching the volume of 1.5 cm³ according to institutional regulations. All the appropriate controls were used.

### Statistics

Statistical parameters (average values, SD, significant differences between groups) were calculated using GraphPad Prism Software. Statistical significance between groups was determined by paired t test or one-way ANOVA with post hoc Tukey's multiple comparison test (p<0.05 was considered statistically significant).

### Example 1: PI3K Inhibition Enhances the Proliferative Ability and Survival of CD8 T Cells by Preserving the Memory Phenotype

T_{CM} CD8 T cells are superior mediators of antitumor immunity than T_{EM} due to their greater proliferative ability. Proliferation of T cells (like many T cell functions) is regulated by the PI3K/Akt pathway. To test the role of PI3K in the differentiation and proliferation of CD8 T cells were tested to determine the effect of the pan PI3K inhibitor GDC-0941 (GDC) on stimulated pMel-1 CD8 T cells activated with 1 μM gp100₂₅₋₃₃.

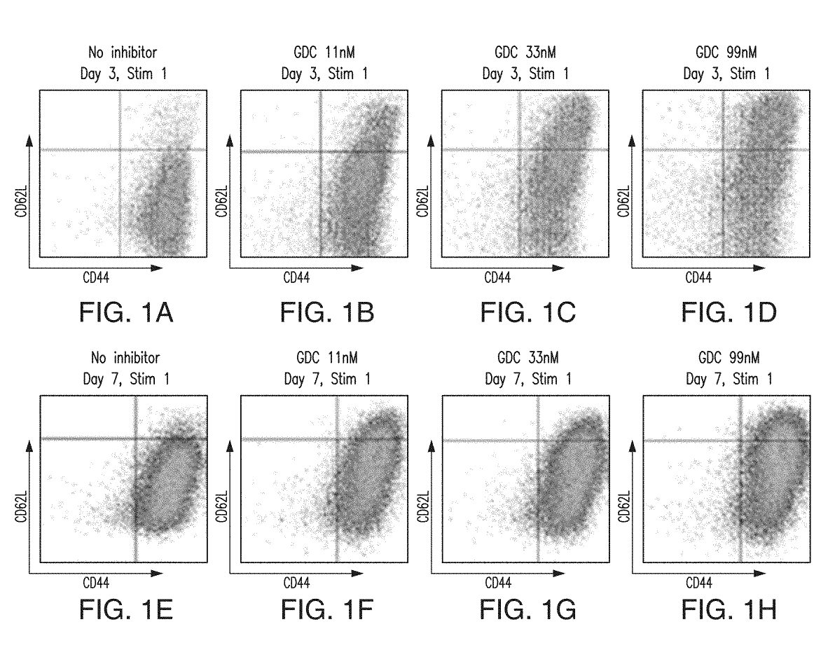

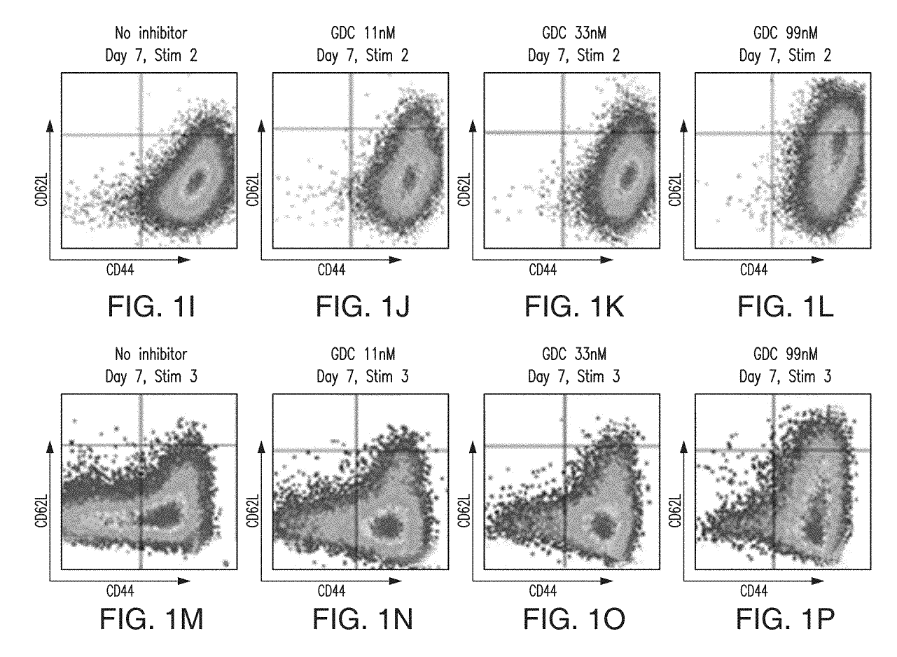

After 3 days of stimulation, it was found that GDC-treated cells consisted of a high percentage of T_{CM} cells (CD62L^{hi}CD44^{hi}), while the majority of non-GDC treated cells were T_{EM} cells (CD62L^{lo}CD44^{hi}). This was observed at all the concentrations used (FIGS. 1A-1P).

In FIGS. 1A-1P, 2A-2D, 3A-3D and 4, non-fractionated splenocytes from pMel-1 mice were stained with VCT and activated with gp100₂₅₋₃₃ peptide (1 μM) in the presence or absence of GDC-0941 (11, 33, 99 and 279 nM). The cells were re-stimulated with gp100₂₅₋₃₃ peptide on days 7, 14 and 21 and their phenotype and proliferation of assessed. The gated cells were viable (7AAD−) CD8+Vβ13+.

In FIGS. 1A-1P, non-GDC treated CD8 T cells are mainly T_{EM} cells (CD62L^{lo}CD44^{hi}), while GDC-treated cells have a high percentage of the T_{CM} phenotype (CD62L^{hi}CD44^{hi}). Terminally differentiated T cells (CD62L^{lo}CD44^{lo}) after the third stimulation are significantly higher in non-treated cells.

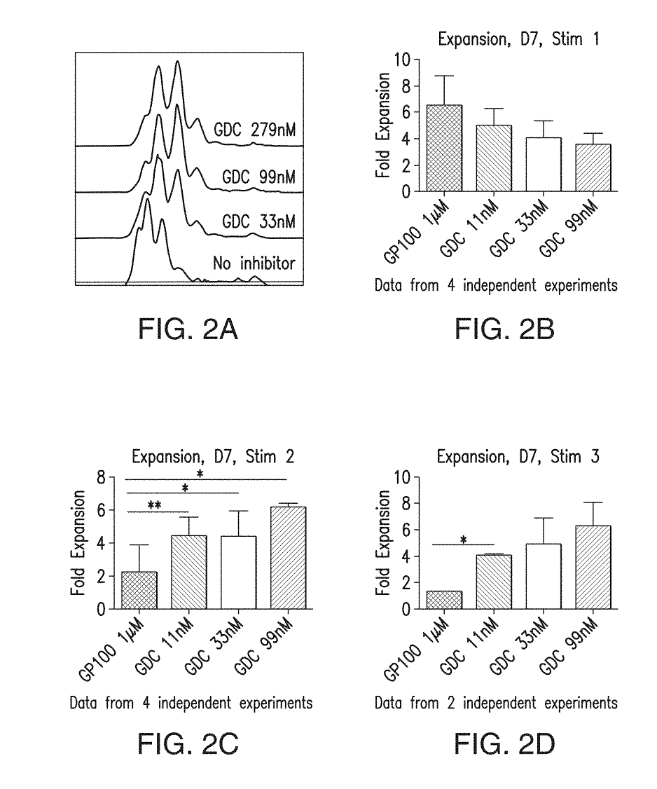

In FIGS. 2B-2D, after 3 days of stimulation, the proliferation of CD8 T cells was inhibited in a dose-dependent manner by GDC-0941 (VCT dilution) (far left). CD8 T cells treated with GDC expand at a significantly high rate with further stimulations. * p<0.05, ** p<0.01.

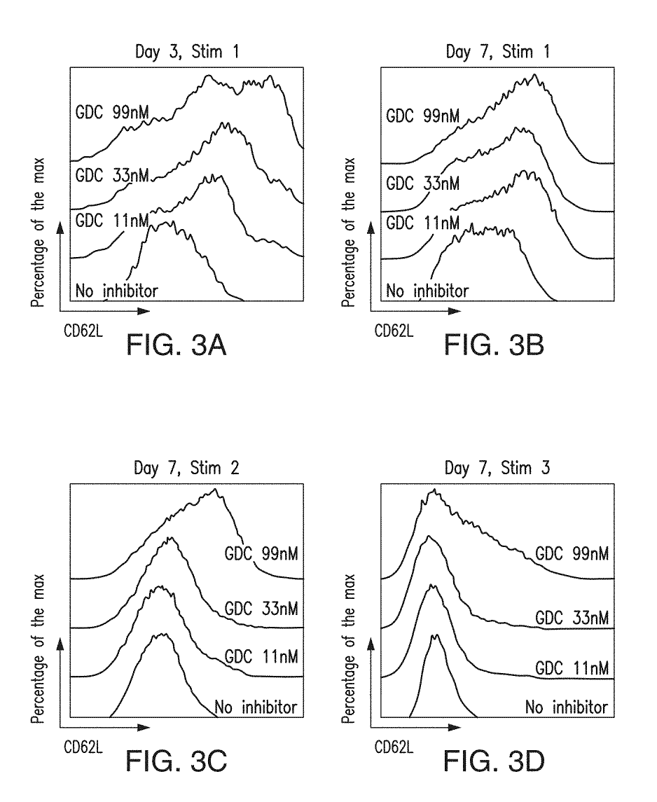

In FIGS. 3A-3D, PI3K inhibition by GDC-0941 maintains a high level of CD62L expression in CD8 T cells on day 3, and on day 7 after each stimulation with gp100.

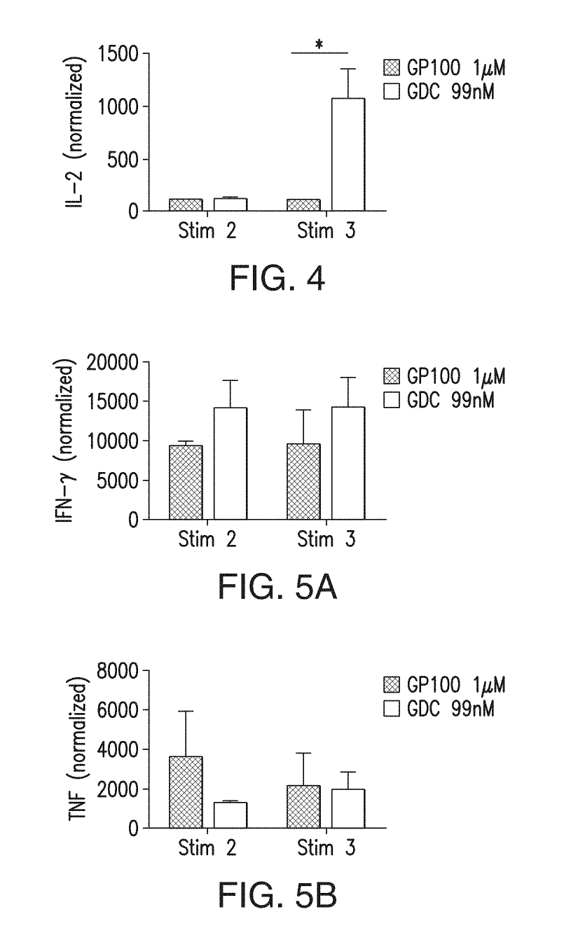

In FIG. 4 GDC-treated CD8 T cells secrete significantly higher levels of IL-2 following stimulation 3, which is consistent with their higher proliferative potential. * p<0.05.

This shows that PI3K inhibition holds the CD8 T cells in the earlier differentiation stages, namely T_{CM}. Remarkably, the same effect was maintained after the second and third stimulation with gp100₂₅₋₃₃ on days 7, 14 and 21 (FIGS. 1A-1P). Furthermore, there was a significantly higher percentage of terminally differentiated effector CD8 T cells)(CD62L^{lo}CD44^{lo} in non-treated cells after consecutive stimulations (FIGS. 1A-1P). Although the percentage of T_{CM} in GDC treated cells, decreased following the second and third stimulations, it was significantly higher than the non-treated cells. This decrease could be attributed to the memory recall of T_{CM} following multiple stimulations; leading to the differentiation of T_{CM} into T_{EM} and effector cells. Additionally, following the third stimulation, there was a higher percentage of T_{EM} in GDC treated cells in comparison to the non-treated cells, emphasizing the effect of PI3K inhibition on delaying the terminal differentiation and maintaining the cells in the earlier differentiation stages.

Taken together, these data show that PI3K inhibition delays terminal differentiation and preserves a reservoir of memory cells (both T_{CM} and T_{EM}), even after several encounters with the antigen.

T_{CM} CD8 T cells possess a greater proliferative ability than T_{EM} upon antigen re-encounter. The proliferation and expansion of CD8 T cells was assessed. As expected, after three days of stimulation, the proliferation and expansion of CD8 T cells treated with GDC was slightly inhibited (FIGS. 2A-2D). At the highest dose tested (279 nM) the inhibitor was found to be toxic, and was therefore used at the lower doses for the rest of the experiments.

Amazingly, with further stimulation, CD8 T cells treated with the PI3K inhibitor expanded at a significantly higher rate than the non-treated cells (FIGS. 2A-2D). Non-treated cells lost the ability to expand following the third encounter with the antigen. These findings show that PI3K inhibition enhanced the cell proliferation and survival of the CD8 T cells.

As can be clearly seen in FIGS. 3A-3D, CD8 T cells treated with GDC maintained high expression levels of CD62L (days 3, 7, 14 and 21), which is a marker associated with high proliferative potential. This correlates with the enhanced proliferation ability of the CD8 T cells observed with the inhibitor treatment.

CD8 T cells' ability to proliferate was further assessed by measuring their ability to secrete IL-2, a property that is diminished in terminally differentiated CD8 T cells. It was found that CD8 T cells treated with GDC maintained a significantly high level of IL-2 secretion even after re-encountering the antigen (FIG. 4).

Taken together, these data show that PI3K inhibition preserves the memory phenotype, hence enhancing the proliferative potential and survival while delaying the terminal differentiation of CD8 T cells.

### Example 2: PI3K Inhibition does not Affect the Ability of CD8 T Cells to Produce Cytotoxic Cytokines and Granzyme B

The data demonstrate that PI3K inhibition enhances proliferation, preserves the T_{CM} phenotype and delays terminal differentiation. To assess the function of T_{CM} cells, their ability to secrete IFN-γ and TNF was assessed.

CD8 T cells from pMel-1 mice were stimulated with gp100₂₅₋₃₃ peptide (1 μM) in the presence or absence of GDC-0941 (99 μM), A66 (288 nM), TGX-221 (45 nM) or CAL-101 (202.5 nM). On days 7 and 14, CD8 T cells were re-stimulated with gp100₂₅₋₃₃ peptide and the IFNγ and TNF levels in the supernatant assessed after 24 hours using CBA. Granzyme B expression was assessed on days 7 and 14.

FIGS. 5A and 5B show that the ability of CD8 T cells to produce IFNγ and TNF was not affected by PI3K inhibition using GDC-0941.

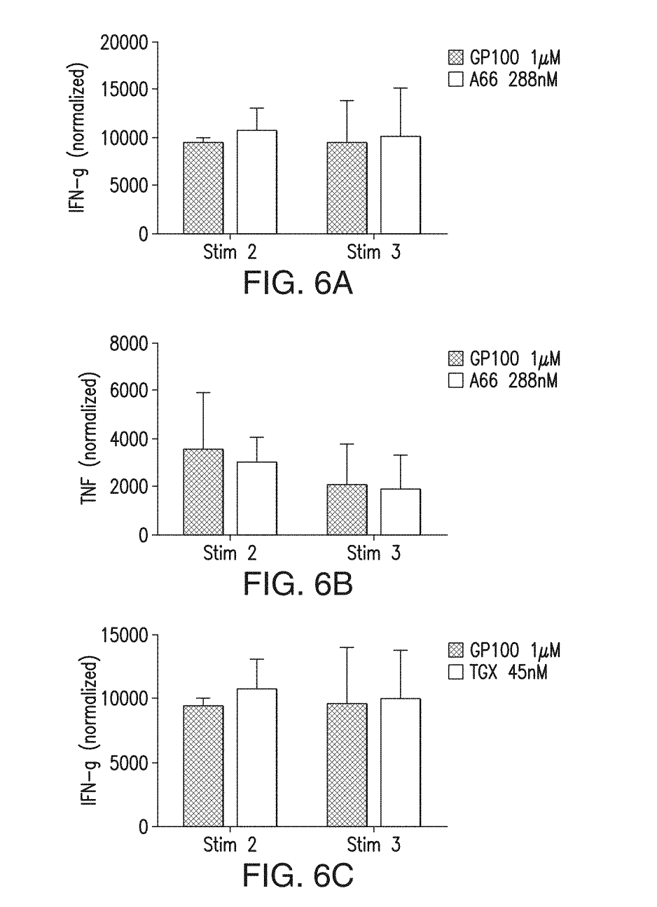

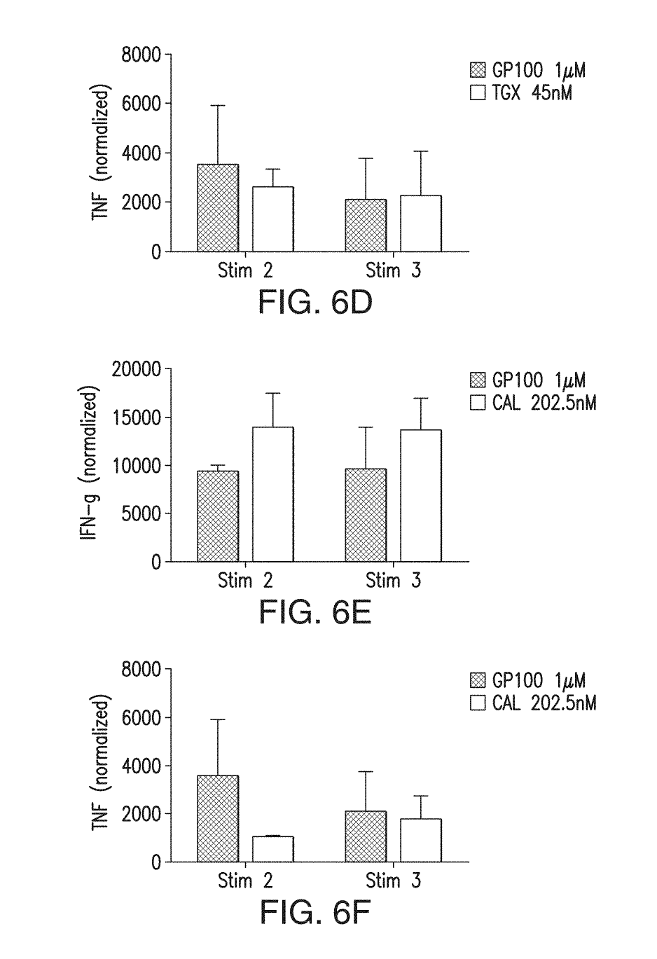

FIGS. 6A-6F show that the ability of CD8 T cells to produce IFNγ and TNF was not affected by the inhibition of specific PI3K isoforms.

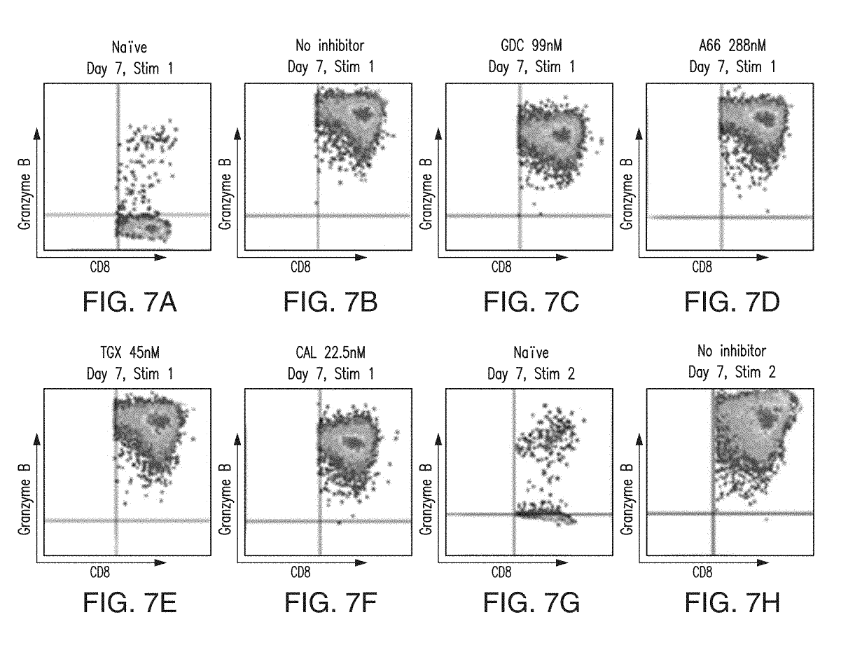

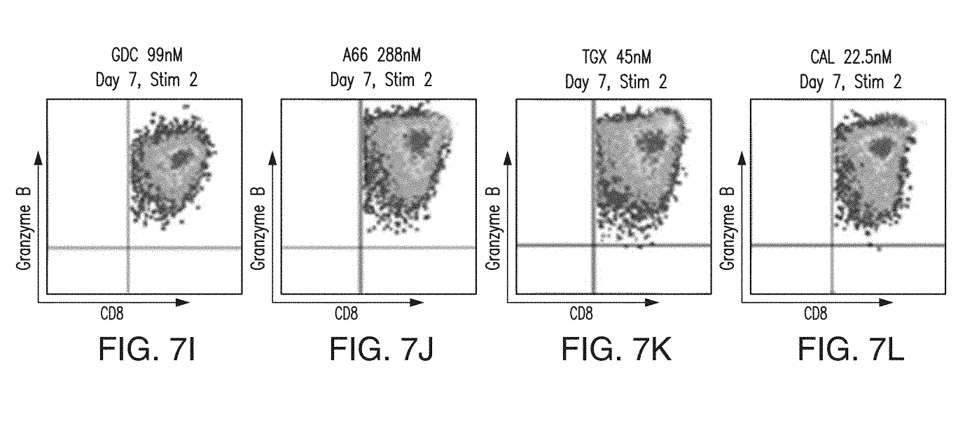

To further test the cytotoxic ability of the CD8 T cells, the level of Granzyme B production was assessed by intracellular staining. Following the first and second stimulations, all the cells (whether treated or non-treated with the inhibitor) produced Granzyme B (in comparison to naïve cells) (FIGS. 7A-7L).

The maintained levels of IFN-γ and TNF secretion suggest that CD8 T cells do not lose their cytotoxic functionality as a result of PI3K inhibition.

### Example 3: PI3K-δ is the Isoform Responsible for Terminal Differentiation of CD8 T Cells

The data show that PI3K inhibition in CD8 T cells delays their terminal differentiation, preserves T_{CM} cells, enhances their proliferative ability while maintaining their cytokine secretion ability and prolonging their survival. The role of specific PI3K isoforms (PI3K-α, PI3K-β and PI3K-δ) in the development, proliferation and function of CD8 T cells is not known. The effect of specific isoform inhibition was assessed using selective inhibitors on CD8 T cells.

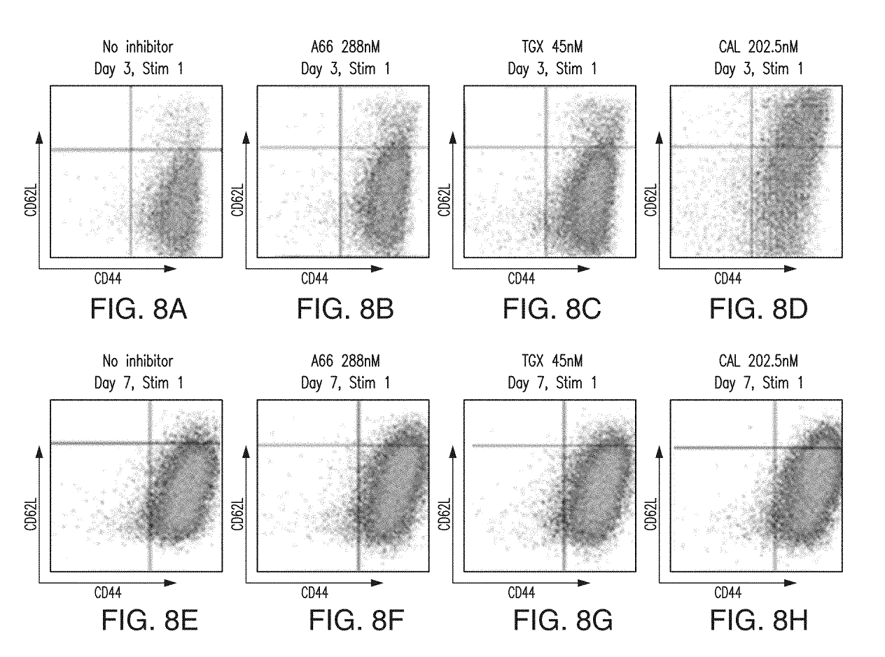

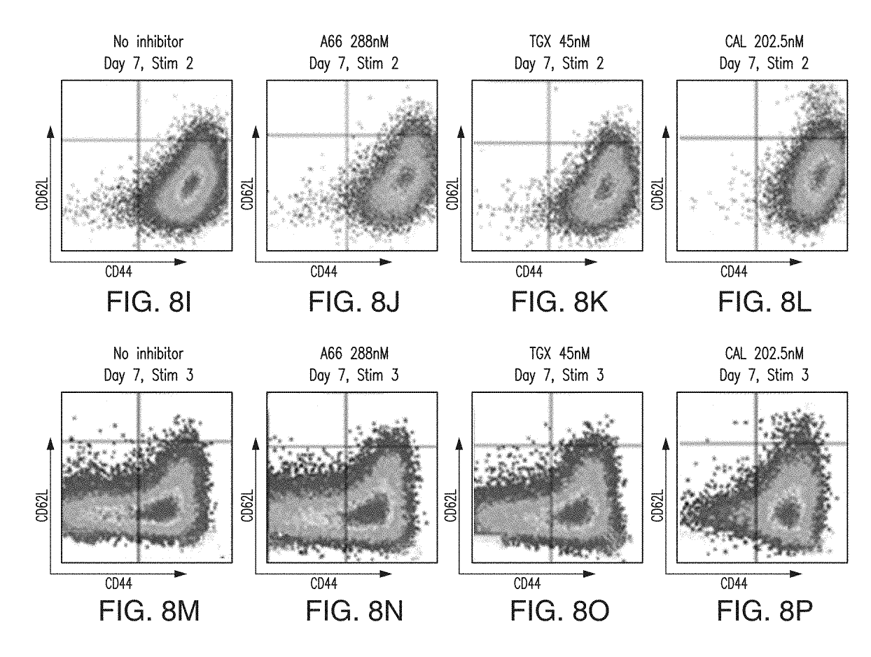

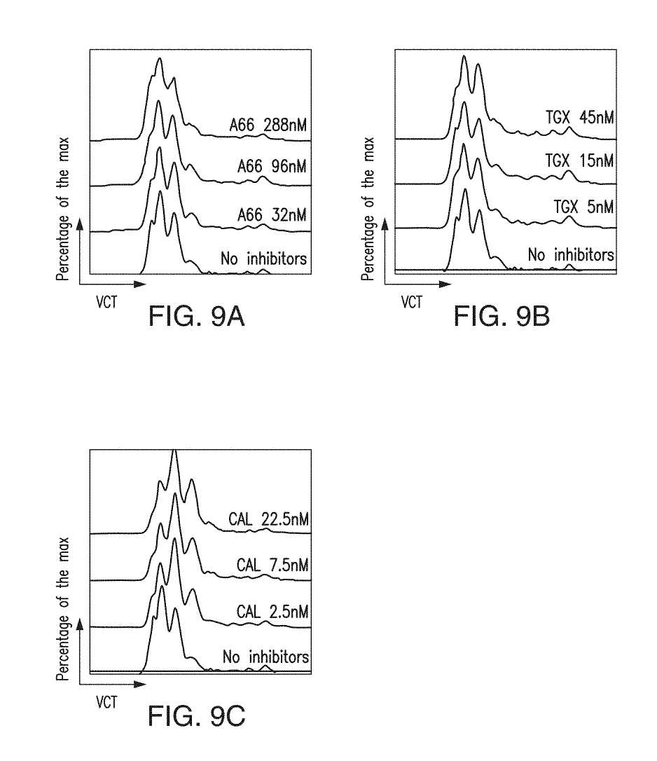

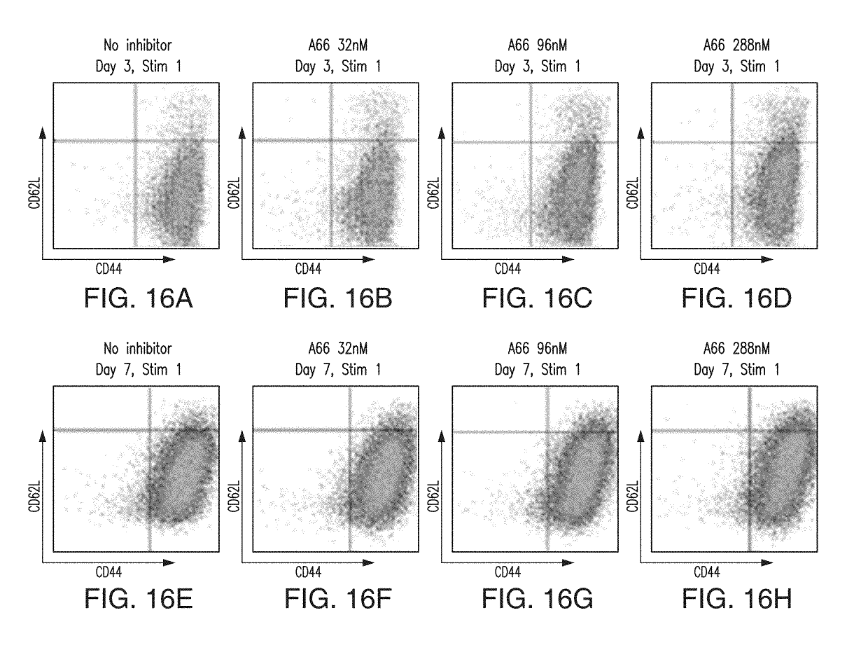

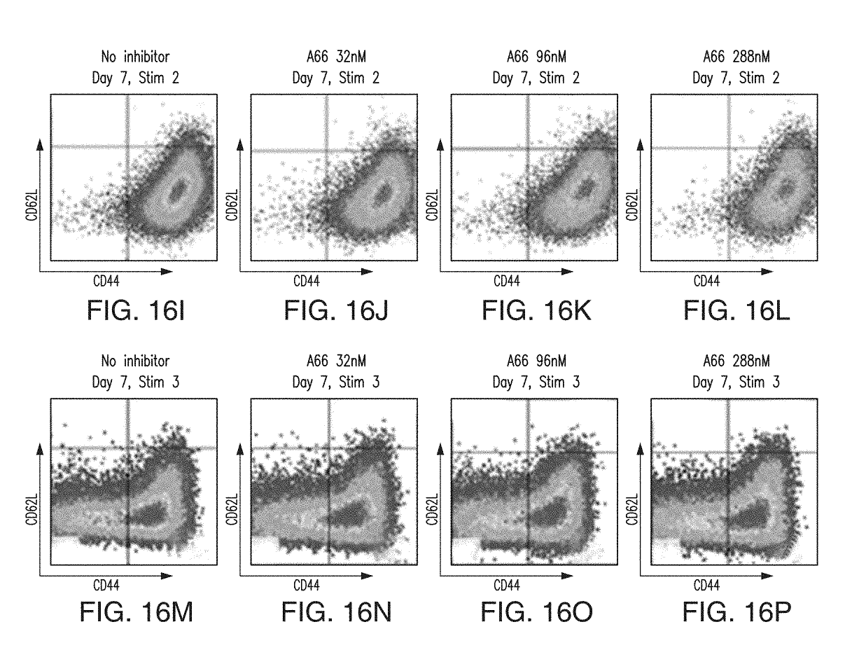

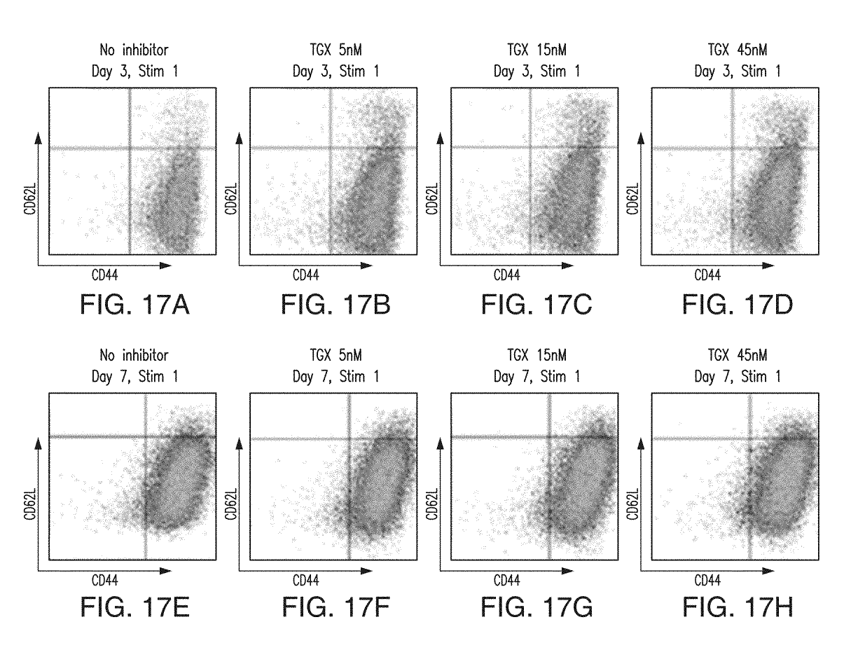

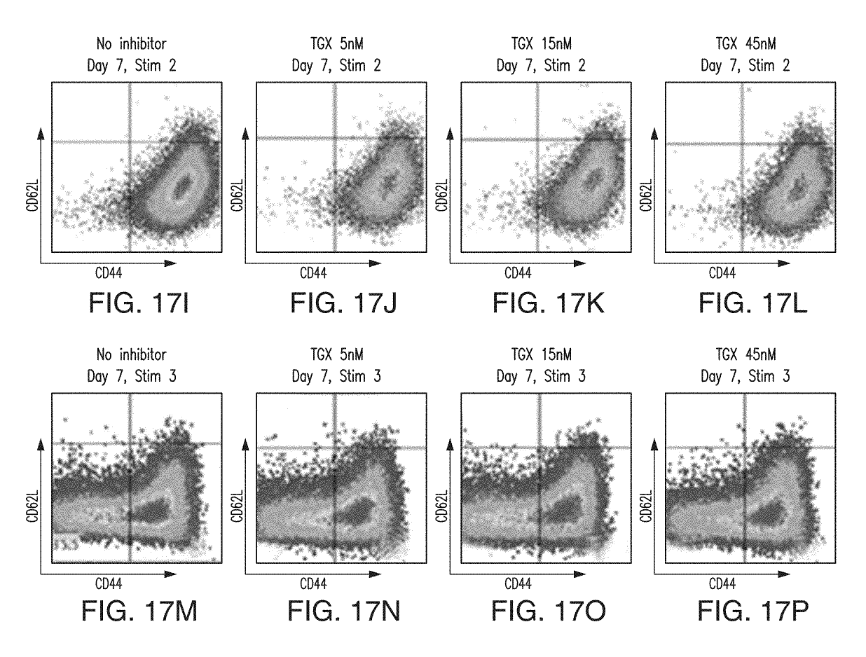

In FIGS. 8A-8D and 9A-9C when the phenotype of the cells was assessed after 3 days of stimulation, CD8 T cells treated with inhibitor s specific for PI3K-α (A66) or PI3K-β (TGX-221), there were no differences in the phenotype of CD8 T cells from the non-treated cells (FIGS. 8A-8C). However, when the PI3K-δ inhibitor (CAL-101) was used (FIG. 8D), CD8 T cells displayed a phenotype similar to that observed with pan PI3K inhibition, where there was a higher percentage of T_{CM} cells when compared to non-treated cells (FIGS. 8A-8P and FIGS. 16A-16P). Although this effect was less prominent after the second and third stimulations, cells treated with CAL-101 displayed a significantly higher percentage of T_{EM} than those treated with the PI3K-α and PI3K-β. Furthermore, the inhibition of PI3K-δ lead to a significantly lower percentage of terminally differentiated CD8 T cells)(CD62L^{lo}CD44^{lo}) (FIGS. 8A-8P, 16A-16P, 17A-17P, and 18A-18P). In FIGS. 8A-8P, non-fractionated splenocytes from pMel-1 mice were activated with gp100₂₅₋₃₃ peptide (1 μM) in the presence or absence of A66 (32, 96 and 288 nM), TGX-221 (5, 15 and 45 nM) or CAL-101 (0.28, 0.83, 2.5, 7.5, 22.5, 67.5 or 202.5 nM). The gated cells were viable (7AAD−) CD8+Vβ13+.

These findings suggest that PI3K-δ is the isoform responsible for terminal differentiation of CD8 T cells and that its inhibition maintains CD8 T cells in earlier stages of differentiation (both T_{CM} and T_{EM}) even after several encounters with the antigen.

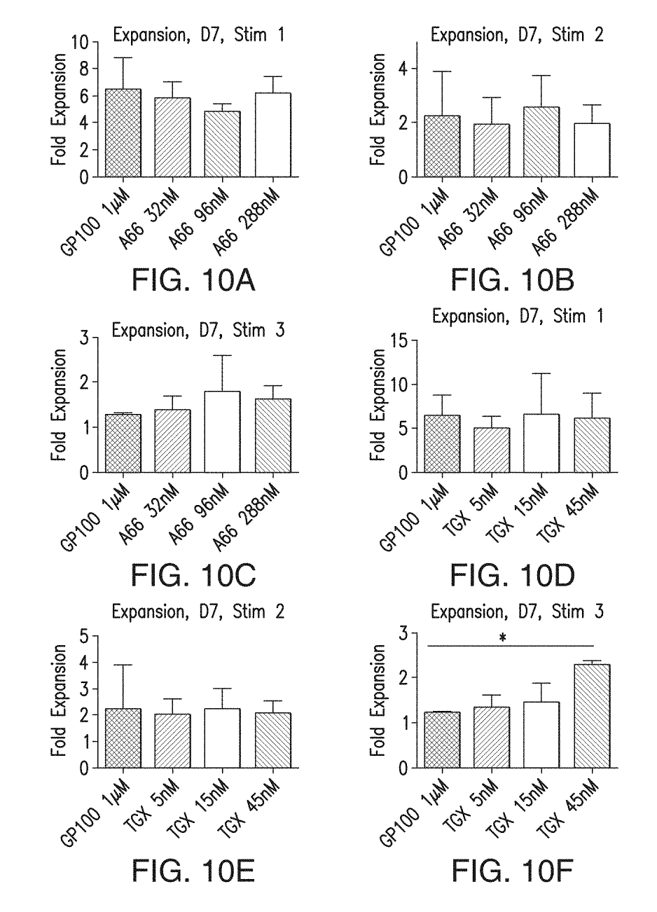

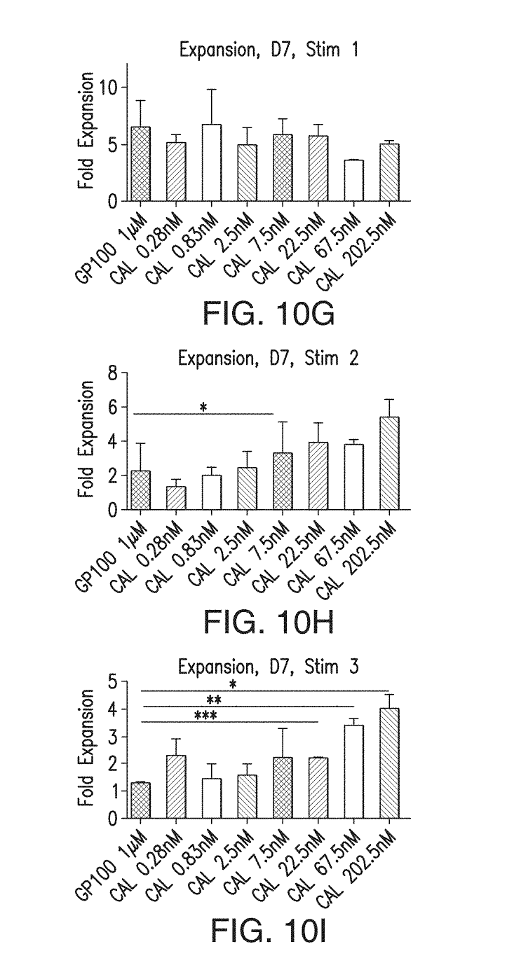

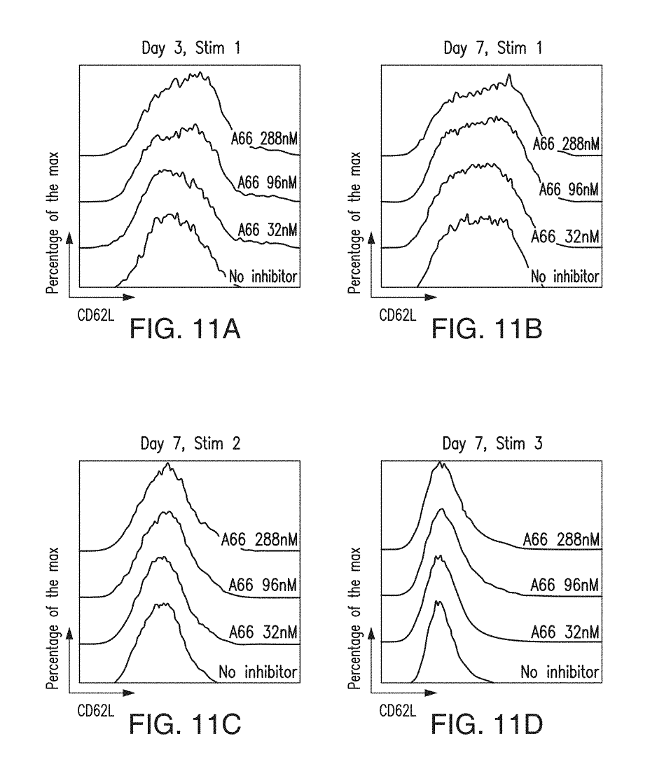

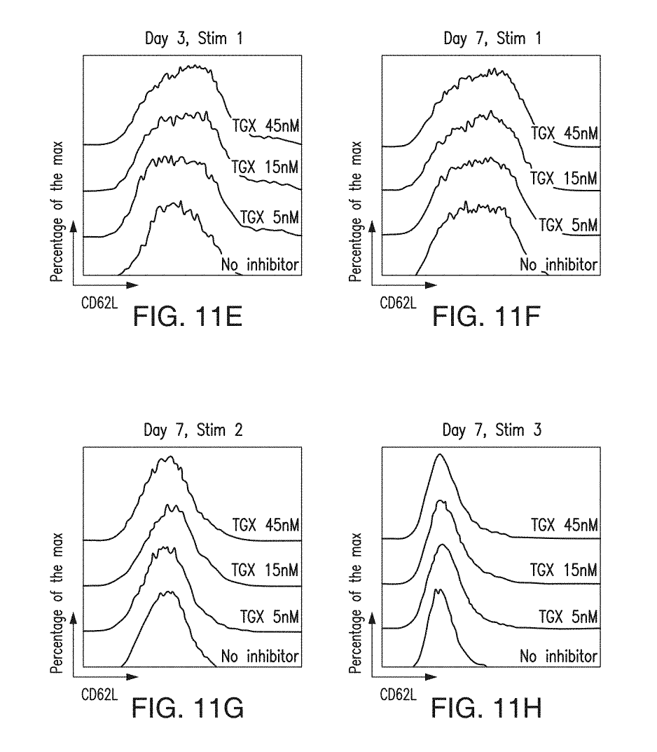

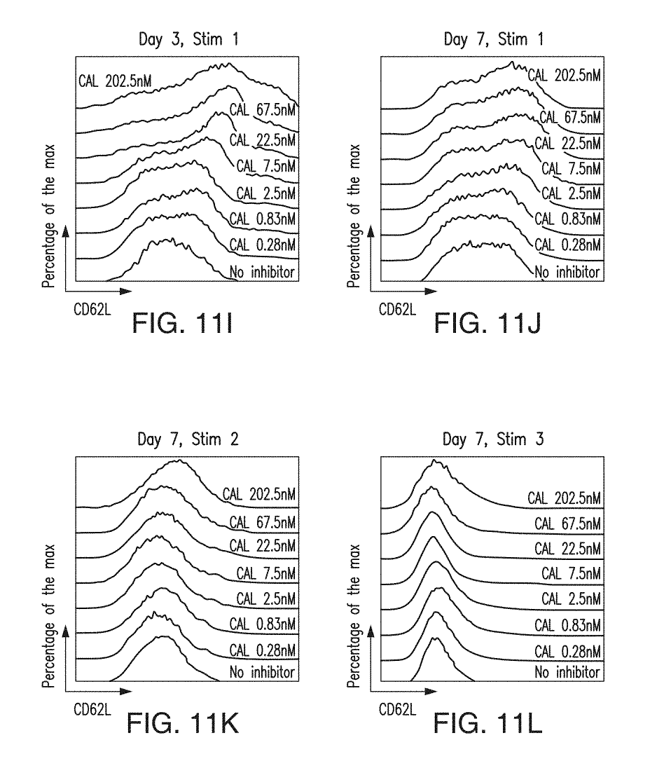

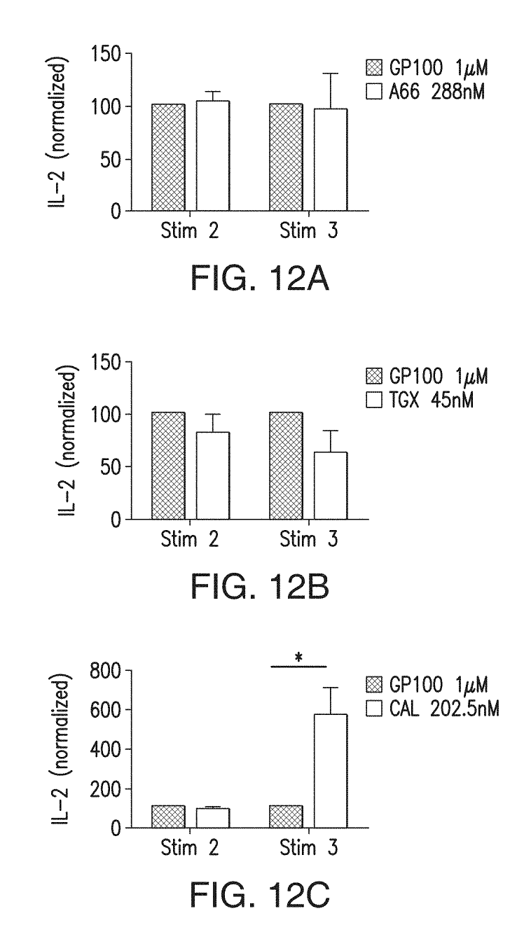

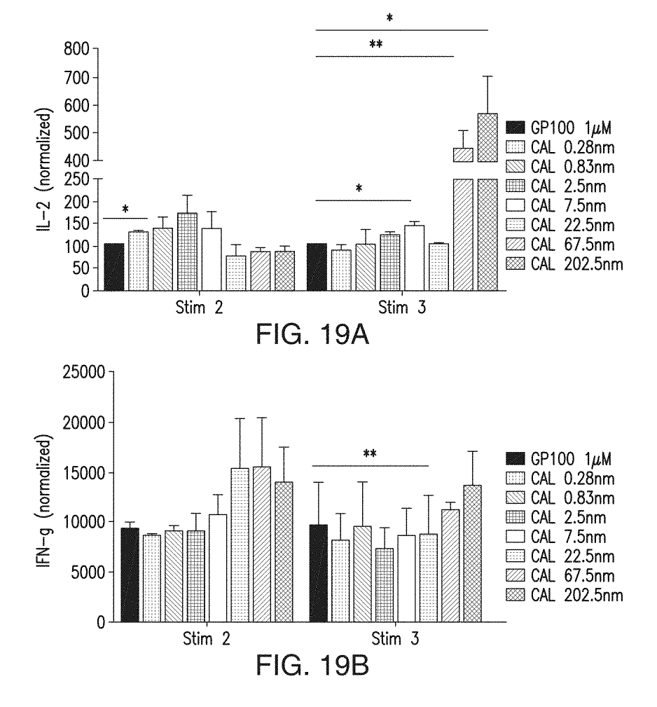

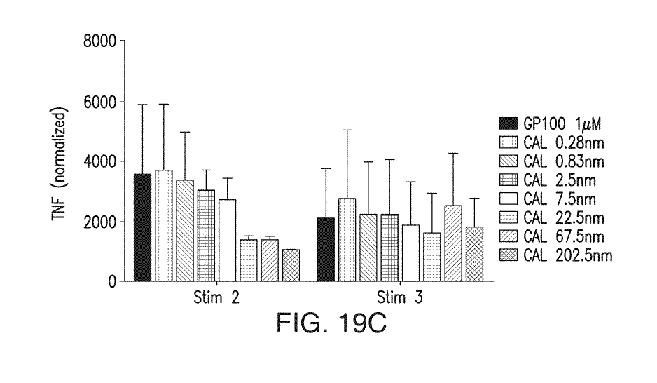

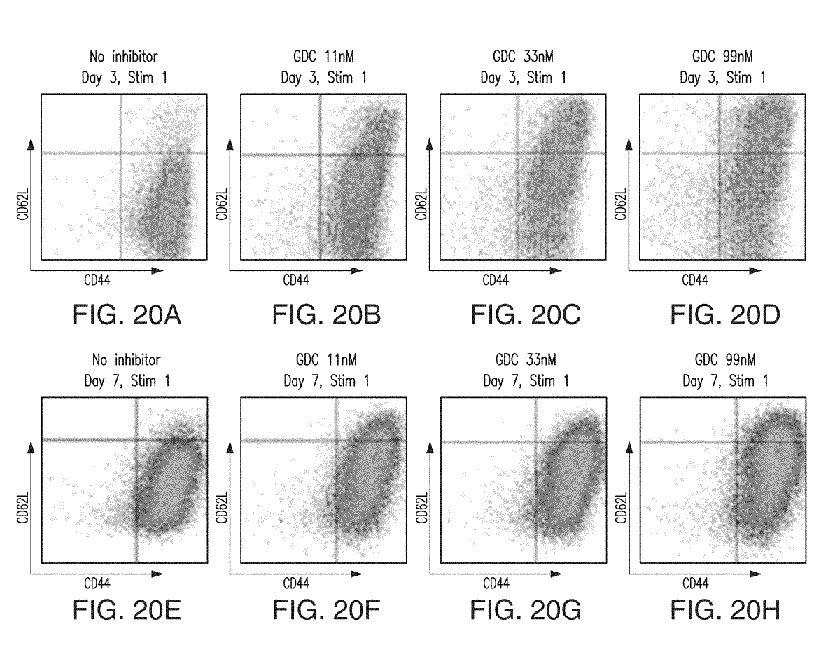

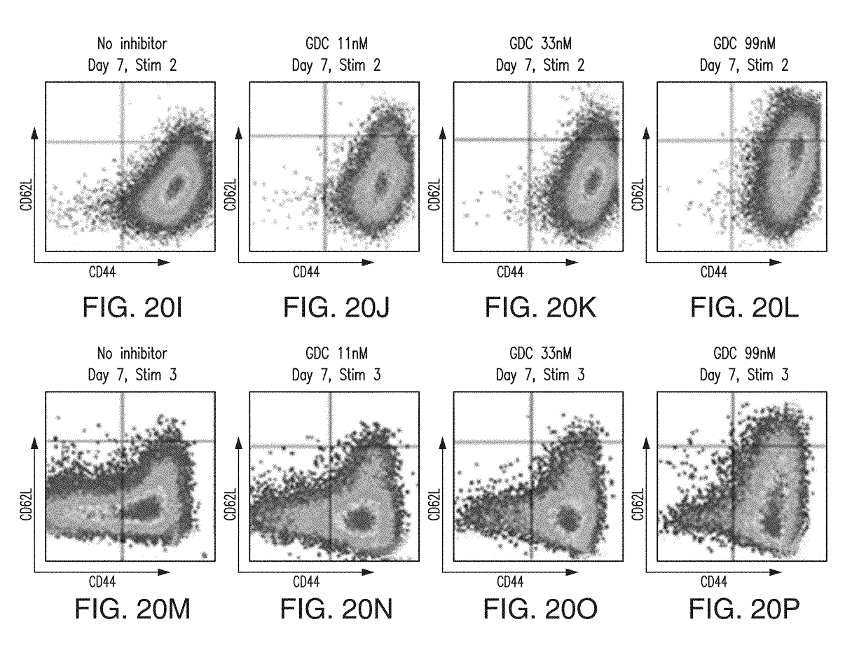

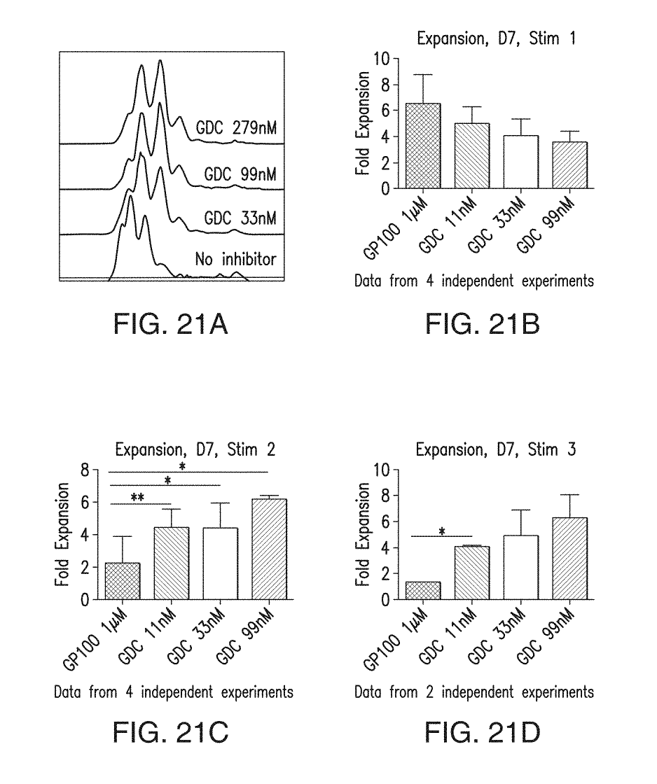

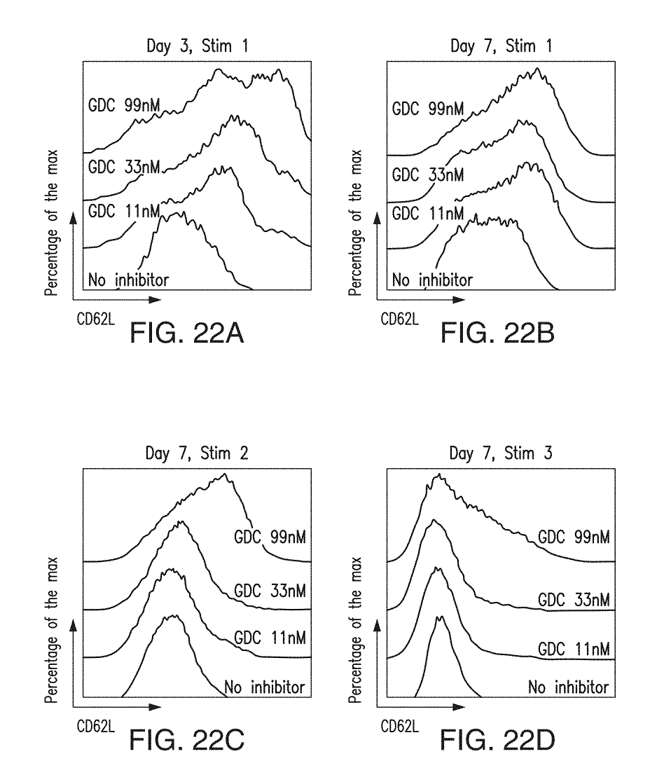

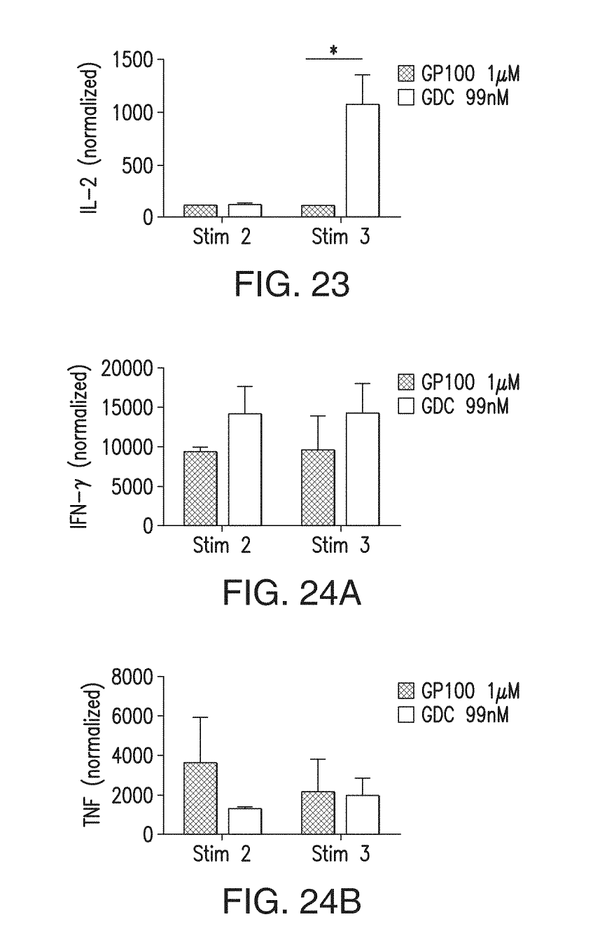

To test if the memory phenotype generated by the inhibition of PI3K-δ possesses an enhanced proliferative ability, the proliferation of CD8 T cells was assessed. It was found that the proliferation of CD8 T cells treated with PI3K-α and PI3K-β inhibitors was not affected. On the other hand, the proliferation of CAL-101 treated cells was marginally inhibited (FIGS. 9A-9C). Additionally, the inhibition of PI3K-δ, but not PI3K-α and PI3K-β significantly enhanced the proliferative ability of CD8 T cells with further stimulations (days 7 and 14) (FIGS. 10A-10I). Treatment of CD8 T cells with the PI3K-δ inhibitor also maintained high expression levels of CD62L (FIGS. 11A-11L) and high secretion levels of IL-2 (FIGS. 12A-C and FIGS. 19A-19C), consistent with the enhanced proliferative ability of the memory CD8 T cells. These high levels of CD62L expression and IL-2 secretion were not observed when CD8 T cells were treated with PI3K-α and PI3K-β inhibitors.

In FIGS. 11A-11L and 12A-12C, non-fractionated splenocytes from pMel-1 mice were stained with VCT and activated with gp100₂₅₋₃₃ peptide (1 μM) in the presence or absence of A66 (32, 96 and 288 nM), TGX-221 (5, 15 and 45 nM) or CAL-101 (0.28, 0.83, 2.5, 7.5, 22.5, 67.5 or 202.5 nM). The cells were re-stimulated with gp100₂₅₋₃₃ on days 7, 14 and 21. The gated cells were viable (7AAD−) CD8+Vβ13+.

Treating CD8 T cells with the different PI3K isoform inhibitors did not affect the cells' ability to produce TNF and IFNγ secretion (FIG. 6A-6FB and FIGS. 19A-19C and Granzyme B (FIG. 7A-7L).

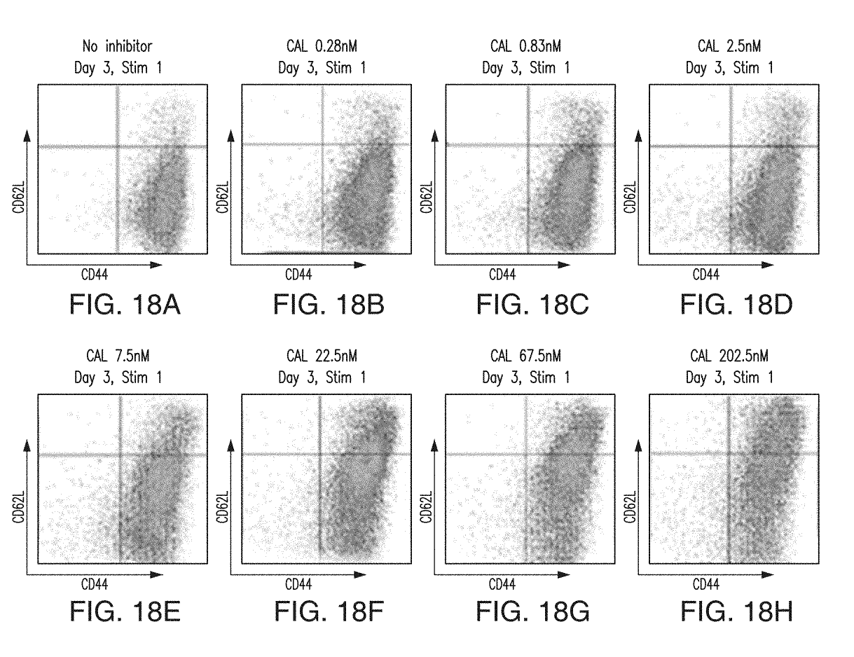

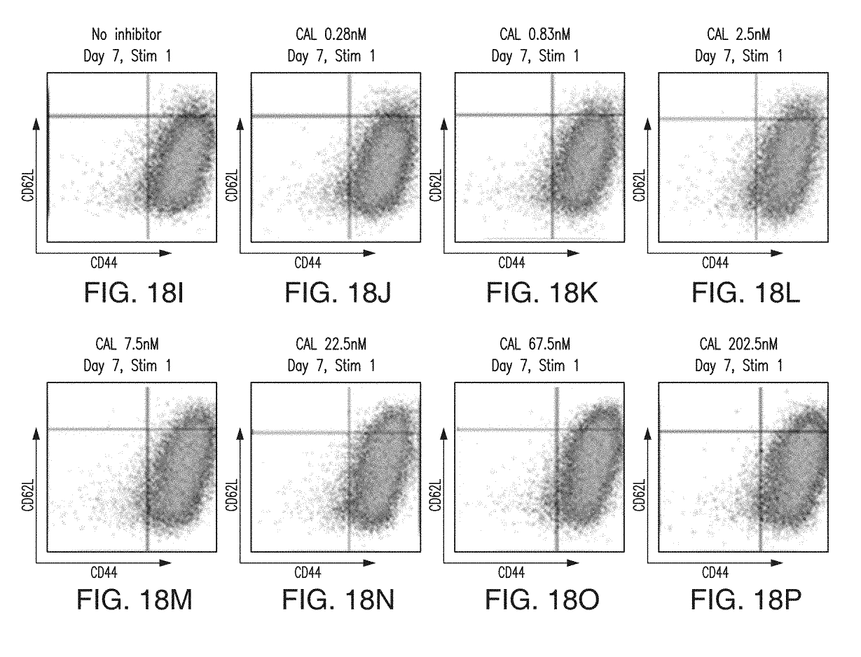

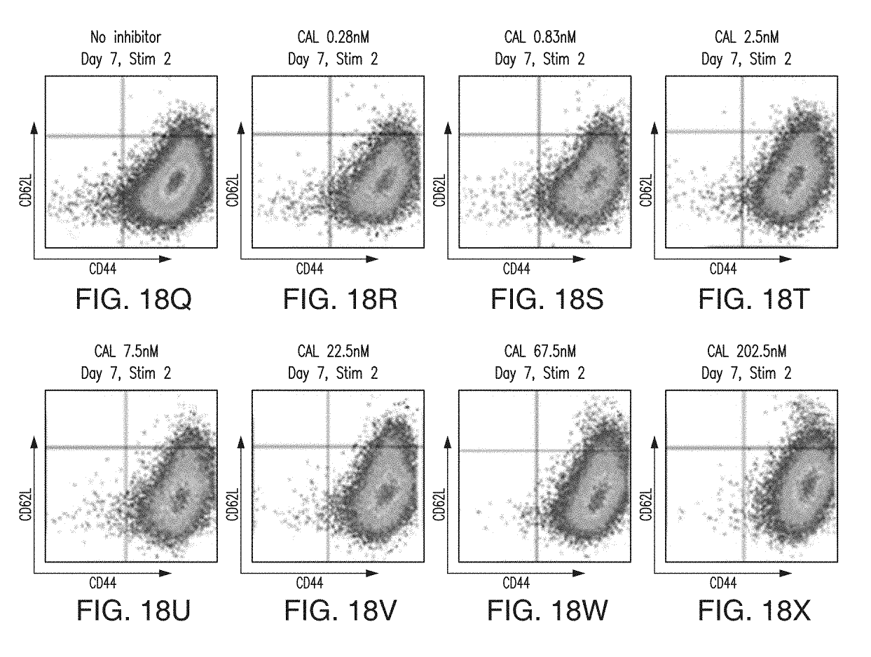

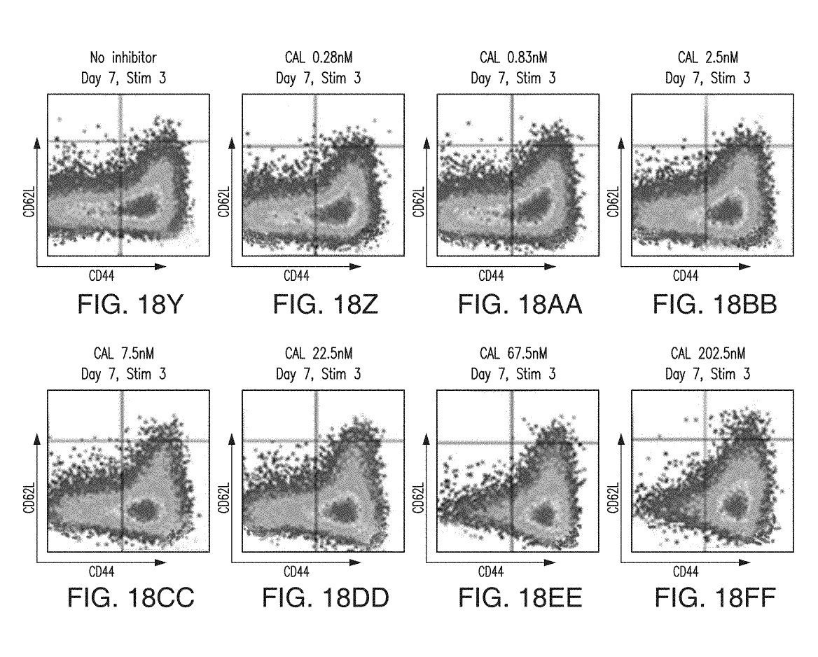

In FIGS. 18A-18FF and 19A-19C non-fractionated splenocytes from pMel-1 mice were activated with gp100₂₅₋₃₃ peptide (1 μM) in the absence or presence of CAL-101 (0.28, 0.83, 2.5, 7.5, 22.5, 67.5 or 202.5 nM). On days 7 and 14, CD8 T cells were re-stimulated with gp100₂₅₋₃₃ peptide and the IFNγ and TNF levels in the supernatant assessed after 24 hours using CBA.

Taken together, the data demonstrate that the inhibition of PI3K-δ, but not PI3K-α or PI3K-β, mitigates the terminal differentiation of CD8 T cells and preserves memory CD8 T cells, thus enhancing their proliferative potential, longevity, and survival without affecting their ability to produce cytokines and Granzyme B.

### Example 4: The Inhibition of PI3K-δ in CD8 T Cells Significantly Enhances their Anti-Tumor Therapeutic Ability In Vivo

The data show that PI3K-δ inhibition delays the terminal differentiation of CD8 T cells and enhances their proliferative ability and survival without affecting their ability to produce cytokines and Granzyme B. To test if these findings translate into enhanced therapeutic ability in vivo tumor antigen specific CD8 T cells were treated with CAL-101 and adoptively transferred into tumor bearing mice and their anti-tumor effect in combination with a tumor specific vaccine was assessed.

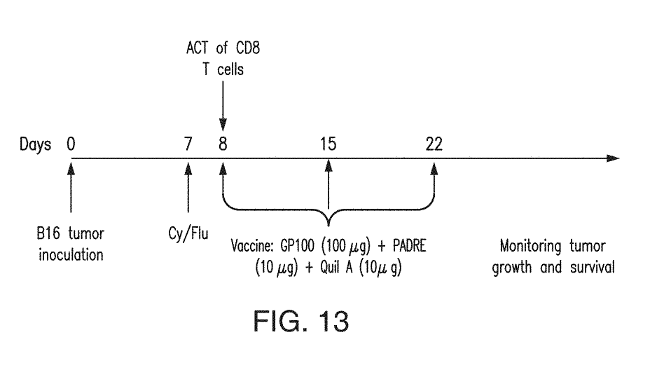

Briefly, pMel-1 cells activated with gp100 with or without CAL-101 were cultured for 7 days and their phenotype assessed (data not shown). Treated cells consisted of a large percentage of T_{CM}. These cells were adoptively transferred into tumor bearing, lymphodepleted mice in combination with gp100 vaccine (administered on days 8, 15 and 22 and corresponding to stimulations 2, 3 and 4) (FIG. 13).

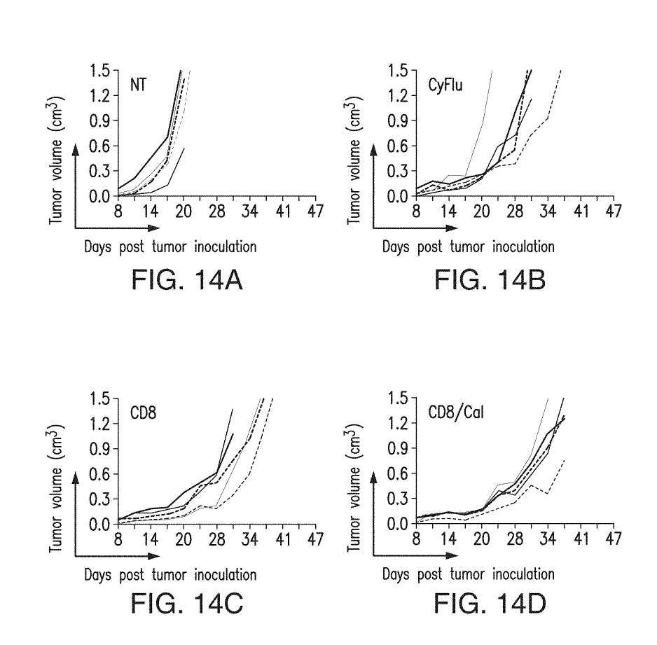

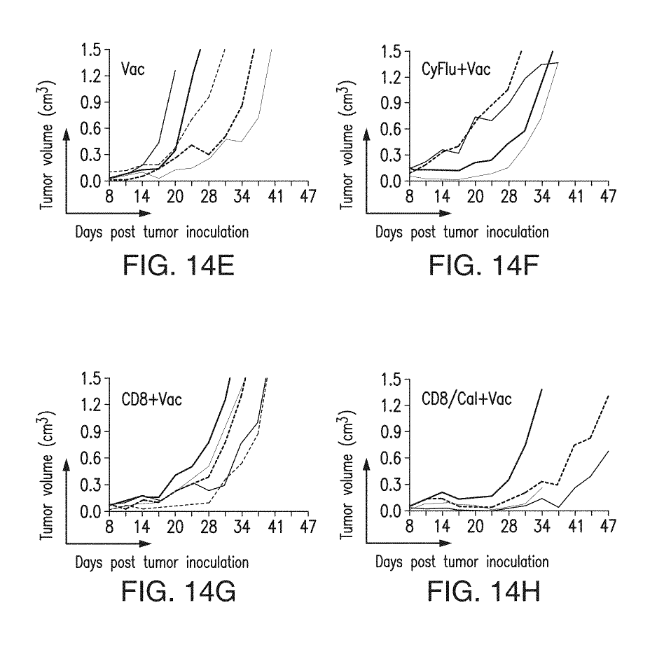

Remarkably, the ACT of CD8 T cells that were activated in vitro in the presence of the PI3K-δ inhibitor CAL-101 greatly slowed down tumor growth in B16 tumor bearing mice. This effect was significantly enhanced when the ACT was combined with the gp100 peptide vaccine. The enhanced therapeutic efficacy was much greater than any other single therapy, including the vaccine, the ACT of non CAL-101-treated CD8 T cells or the combination of both (FIGS. 14A-14H).

In FIGS. 13, 14A-14H, and 15, mice were implanted with B16 in the right flank on day 0. On day 7, mice were lymphodepleted with CyFlu and on Day 8, 1 million CD8 T cells from pMel-1 mice cultured in the presence or absence of CAL-101 were adoptively transferred. The appropriate groups were vaccinated with gp100/PADRE/Quil A vaccine on day 8, 15 and 22. Animal survival and tumor growth was monitored. NT-no treatment, Vac-vaccine, Cyflu-cycophosphamide/fludarabine, CD8/CAL-CD8 T cells treated with CAL-101. All mice that received ACT were lymphodepleted with Cy/Flu.

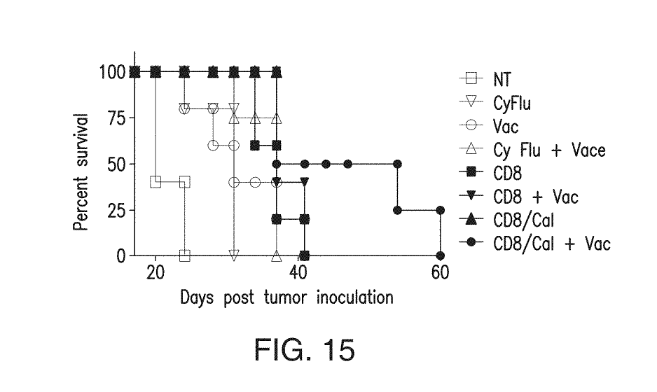

Furthermore, the combination of ACT of CAL-101 treated CD8 T cells with the vaccine greatly prolonged the animal survival (FIG. 15). Similar results were obtained when treatment was started at a later date with larger tumors (data not shown).

These data clearly demonstrate the superior anti-tumor functionality of CD8 T cells treated with a PI3K-δ inhibitor. This can be attributed to the enhanced proliferative ability, longevity, survival and maintenance of the memory phenotype.

### Example 5: PI3K Inhibition Preserves the Memory Phenotype and Enhances the Proliferative Ability of CD8 T-Cells

Non-fractionated splenocytes from pMel-1 mice were stained with VCT and activated with gp100₂₅₋₃₃ peptide (1 μM) in the presence or absence of GDC-0941 (11, 33, 99 and 279 nM). The cells were re-stimulated with gp100₂₅₋₃₃ on days 7 and 14 and their phenotype and proliferation assessed. Gated cells were viable (7AAD−) CD8+Vβ13+.

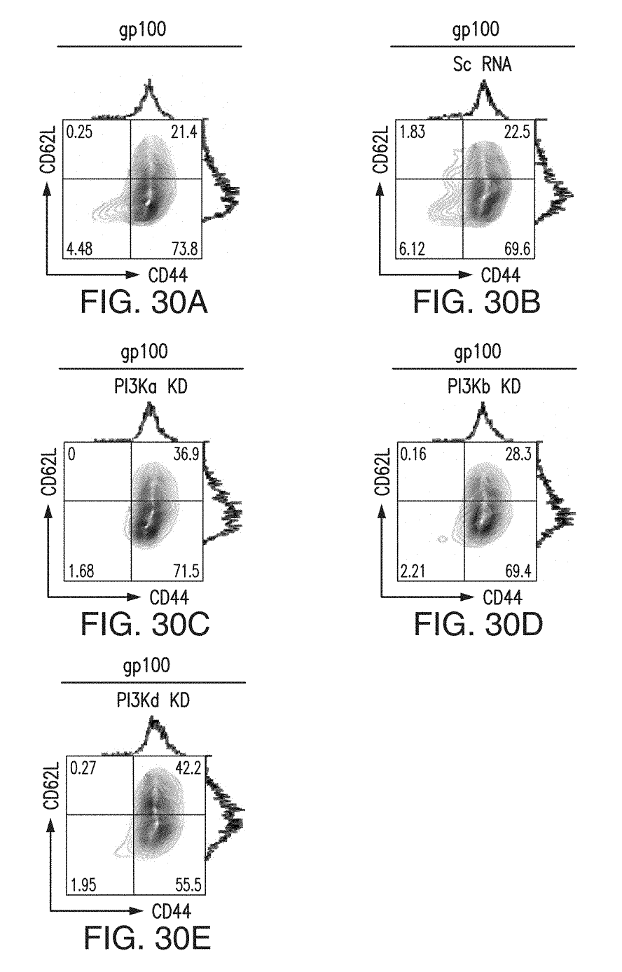

FIGS. 30A-30P show non-GDC treated CD8 T-cells are mainly T_{EM} cells (CD62L^{lo}CD44^{hi}) (96%), while GDC-treated cells have a high percentage (37% at the highest concentration) of T_{CM} phenotype (CD62L^{hi}CD44^{hi}). Terminally differentiated T-cells)(CD62L^{lo}CD44^{lo}) after the third stimulation are significantly higher in non-treated cells (36%) compared to only 4% with the highest GDC concentration. T_{CM} and T_{EM} were maintained with GDC treatment after the third stimulation (3.5 and 93% respectively), compared to only less than 0.01 and 64% in the non-treated cells.

FIG. 30B shows that after 3 days of stimulation, the proliferation of CD8 T-cells was inhibited in a dose-dependent manner by GDC-0941 (VCT dilution) (far left). CD8 T-cells treated with GDC expand at a significantly high rate with further stimulations.* p<0.05, ** p<0.01.

FIG. 30C shows that PI3K inhibition by GDC-0941 maintains a high level of CD62L expression in CD8 T-cells on day 3, and on day 7 after each stimulation with gp100.

FIG. 30D shows that GDC-treated CD8 T-cells secrete significantly higher levels of IL-2 following stimulation 3, which is consistent with their higher proliferative potential. Data normalized to GP100, * p<0.05.

### Example 6: PI3K Inhibition does not Affect the Secretion of IFNγ and TNF

CD8 T-cells from pMel-1 mice were stimulated with gp100₂₅₋₃₃ peptide (1 μM) in the presence or absence of GDC-0941 (99 μM), A66 (288 nM), TGX-221 (45 nM) or CAL-101 (202.5 nM). On days 7 and 14, CD8 T-cells were re-stimulated with gp100₂₅₋₃₃ peptide and the IFNγ and TNF levels in the supernatant assessed after 24 hours using CBA. Granzyme B expression was assessed on days 7 and 14.

FIGS. 24A-24B show the ability of CD8 T-cells to produce IFNγ and TNF was not affected by PI3K inhibition using GDC-0941.

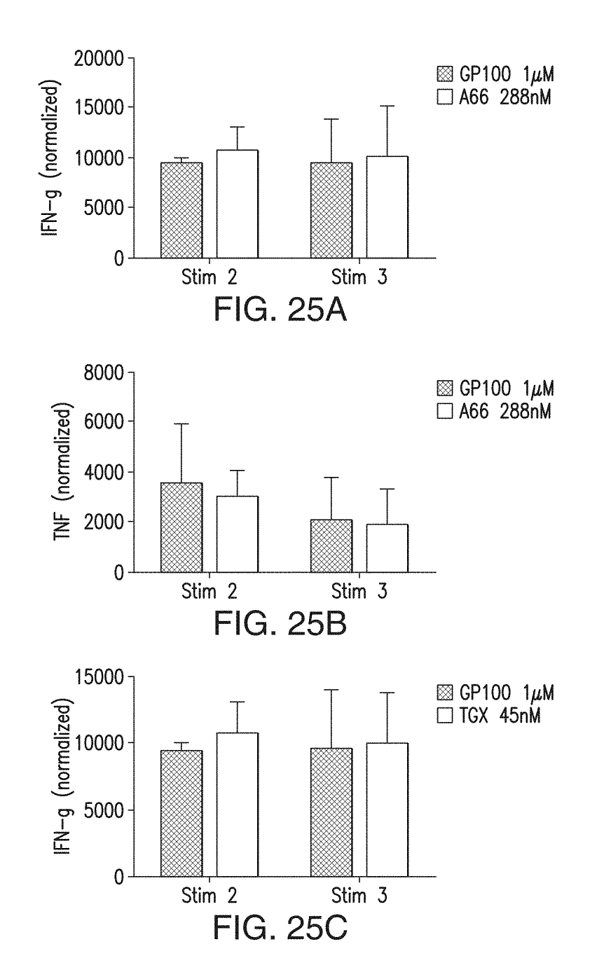

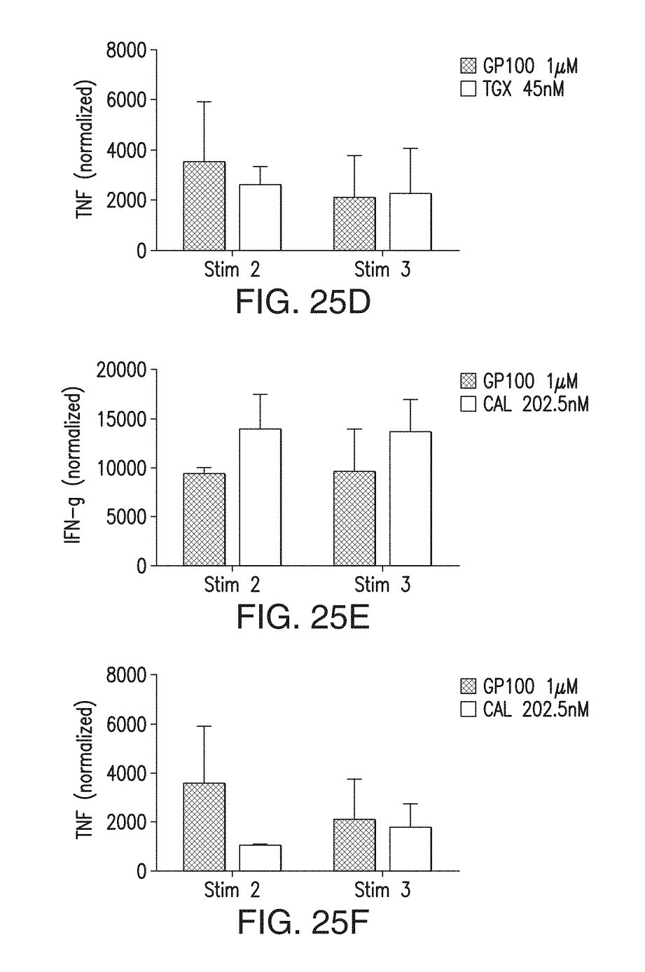

FIGS. 25A-25F show the ability of CD8 T-cells to produce IFNγ and TNF was not affected by the inhibition of specific PI3K isoforms.

### Example 7: The Inhibition of PI3K-δ, but not PI3K-α or PI3K-β, Preserves Memory Cells and Enhances the Proliferative Ability of CD8 T-Cells

Non-fractionated splenocytes from pMel-1 mice were stained with VCT and activated with gp100₂₅₋₃₃ peptide (1 μM) in the presence or absence of A66 (32, 96 and 288 nM), TGX-221 (5, 15 and 45 nM) or CAL-101 (0.28, 0.83, 2.5, 7.5, 22.5, 67.5 or 202.5 nM). The cells were re-stimulated with gp100₂₅₋₃₃ on days 7, 14 and 21. Gated cells were viable (7AAD−)CD8+Vβ13+.

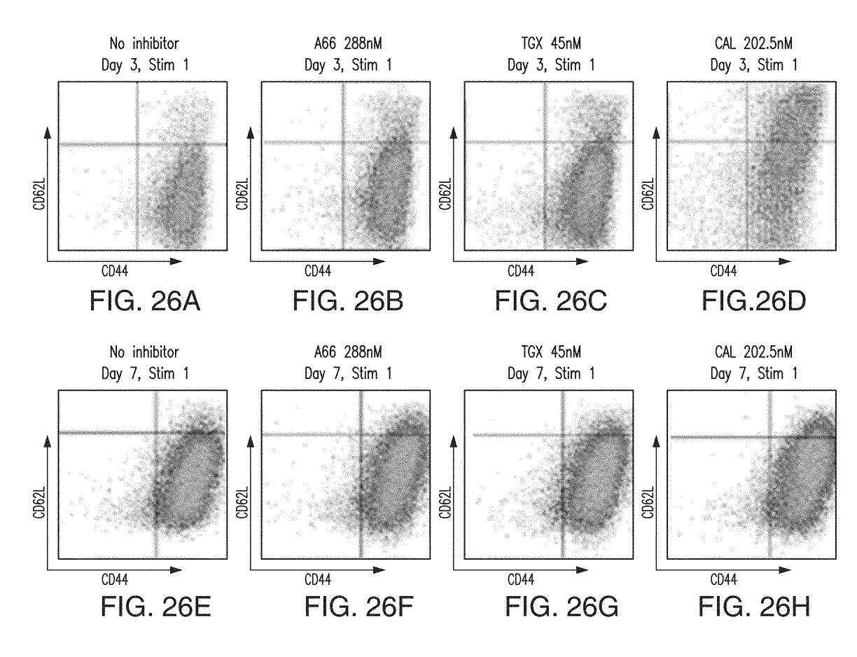

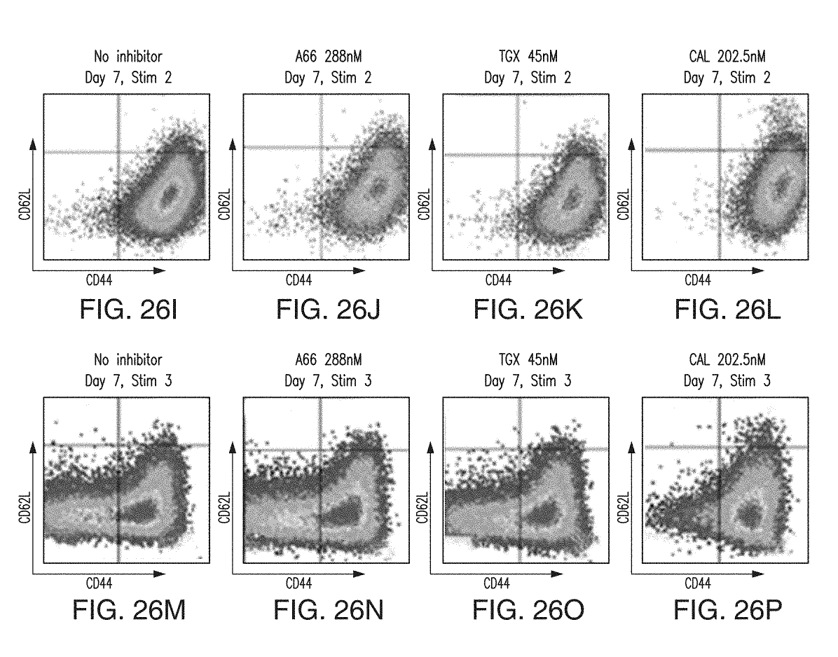

FIGS. 26A-26P show PI3K-δ inhibition, but not PI3K-α or PI3K-β preserves the memory phenotype. In this representative example, A66 and TGX treated cells have a similar profile to non-treated cells, while CAL-101 treated cells have a significantly higher percentage of T_{CM} (CD62L″CD44″) (32% compared to 4%, D3, stim1) and T_{EM} cells (CD62L^{lo}CD44^{hi}) (95% compared to 64%, D7, Stim3), and a lower percentage of terminally differentiated T-cells (CD62L^{lo}CD44¹⁰) (5% compared to 36%, D7, Stim 3).

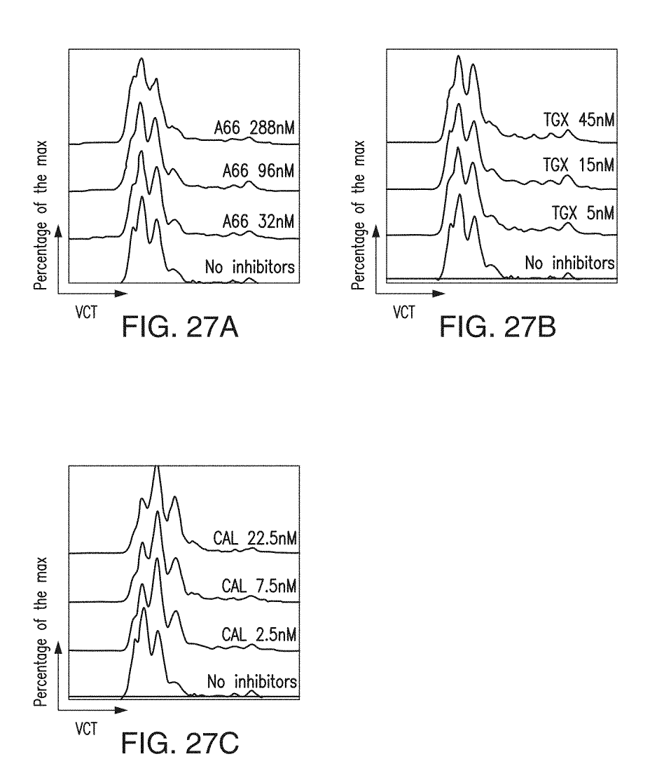

FIGS. 27A-27C show the proliferation of CD8 T-cells is marginally inhibited by PI3K-δ inhibition, but not PI3K-α or PI3K-β (day3).

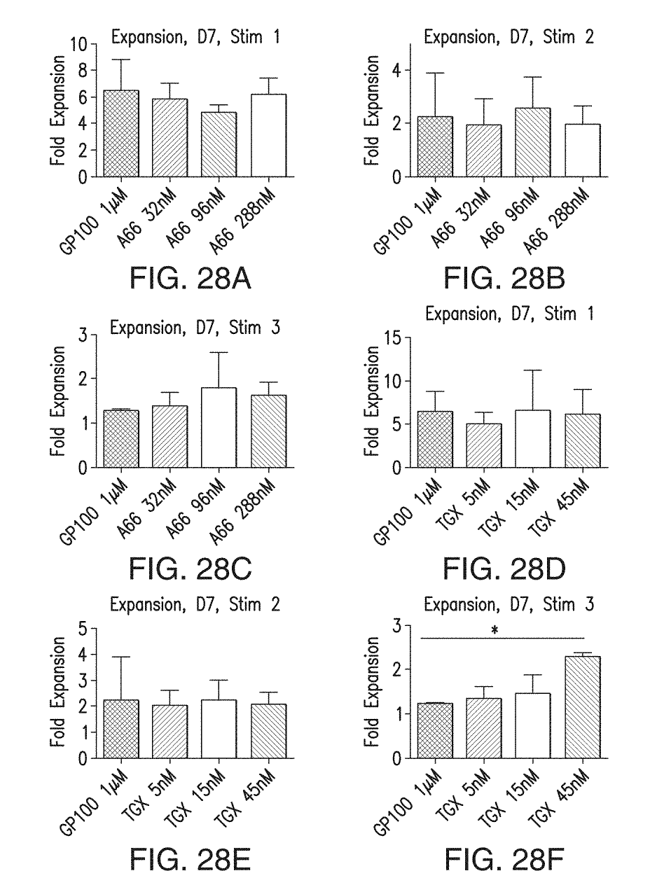

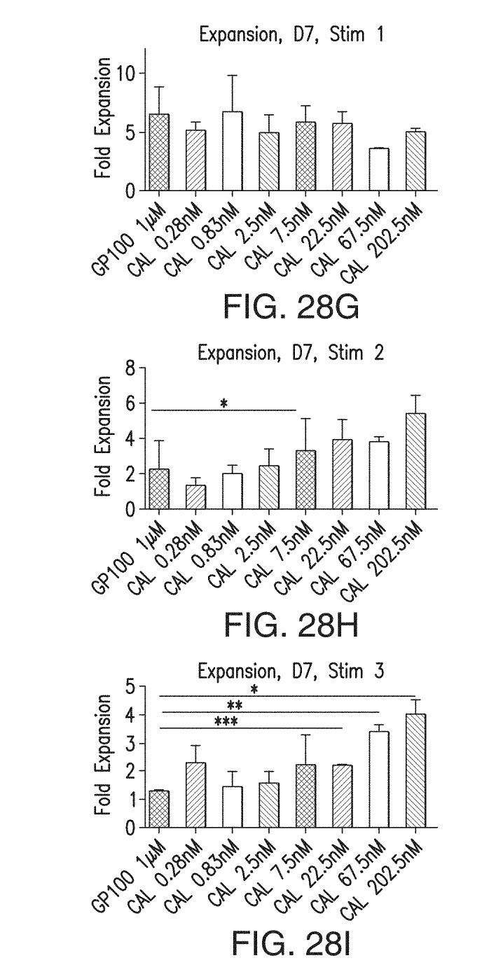

FIGS. 28A-28I show the expansion of CD8 T-cells treated with the inhibitor is significantly enhanced with further stimulations with PI3K-δ inhibition, but not PI3K-α or PI3K-β. * p<0.05, ** p<0.01, *** p<0.001.

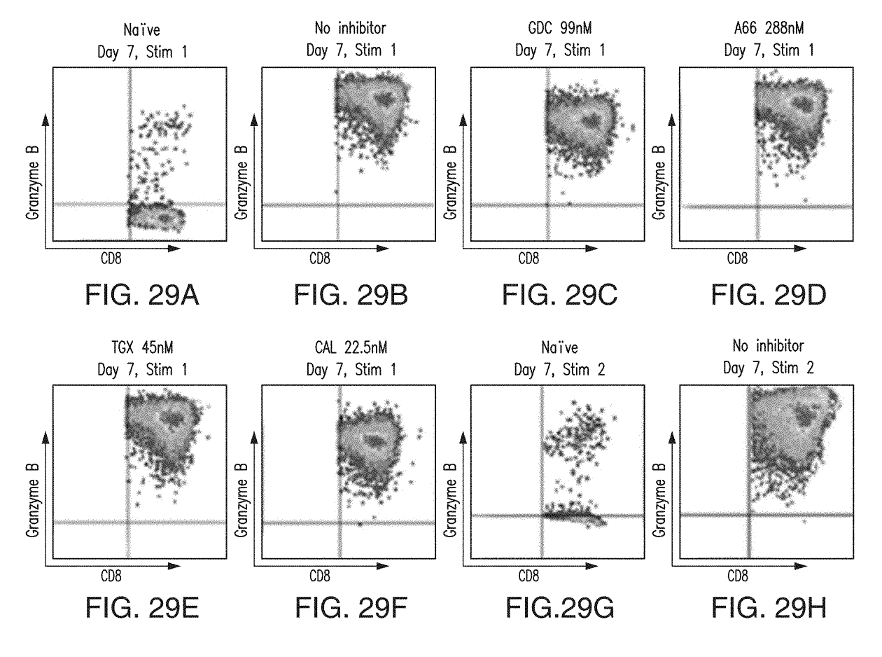

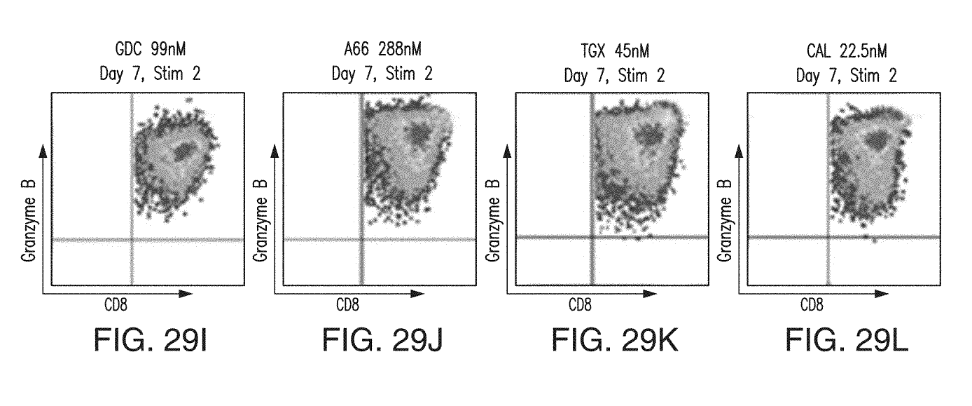

FIGS. 29A-29L show the ability of CD8 T-cells to produce Granzyme B was not affected by PI3K inhibition.

FIGS. 30A-30E show knockdown of PI3K-δ, but not PI3K-α or PI3K-β, preserves memory cells. CD8 cells from pMel-1 mice were activated with gp100₂₅₋₃₃ peptide (1 μM) in the presence or absence of siRNA for PI3Ka, PI3Kb or PI3Kd. PI3K-δ inhibition, but not PI3K-α or PI3K-β preserves the memory phenotype.

### Example 8: The Inhibition of PI3K-δ, but not PI3K-α or PI3K-β, Preserves High Expression Levels of CD62L and High Secretion of IL-2 in CD8 T-Cells

Non-fractionated splenocytes from pMel-1 mice were stained with VCT and activated with gp100₂₅₋₃₃ peptide (1 μM) in the presence or absence of A66 (32, 96 and 288 nM), TGX-221 (5, 15 and 45 nM) or CAL-101 (0.28, 0.83, 2.5, 7.5, 22.5, 67.5 or 202.5 nM). The cells were re-stimulated with gp100₂₅₋₃₃ on days 7, 14 and 21. Gated cells were viable (7AAD−)CD8+Vβ13+.

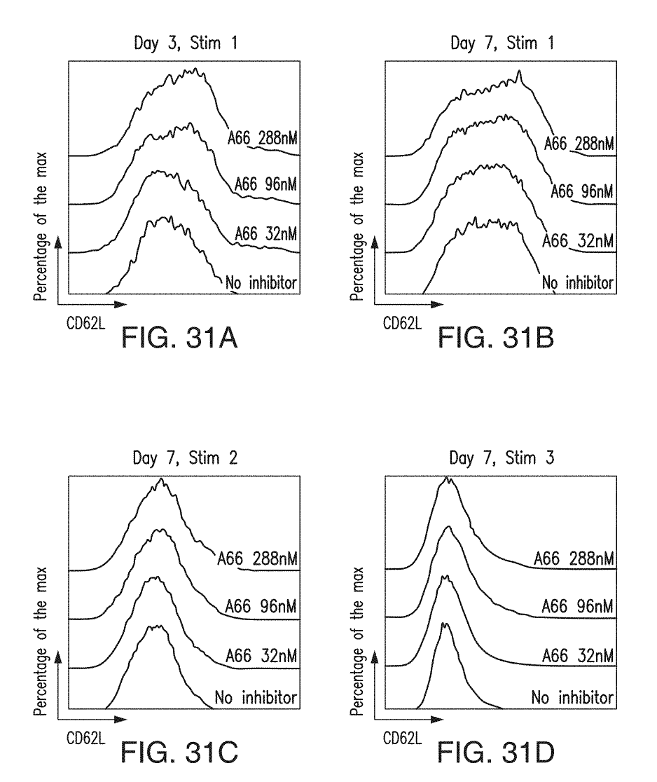

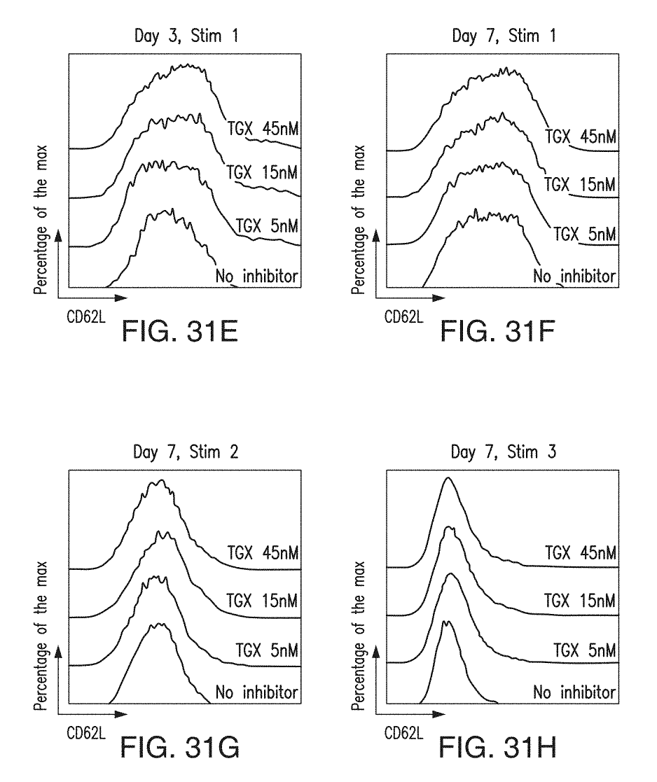

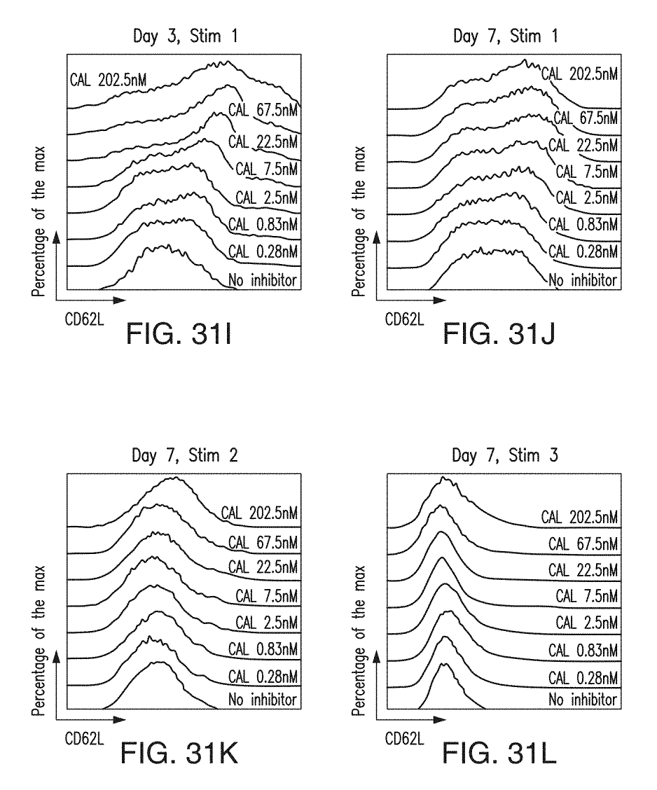

FIGS. 31A-31L show PI3K-δ, but not PI3K-α or PI3K-β maintains a high level of CD62L expression in CD8 T-cells on day 3, and on day 7 after each stimulation with gp100.

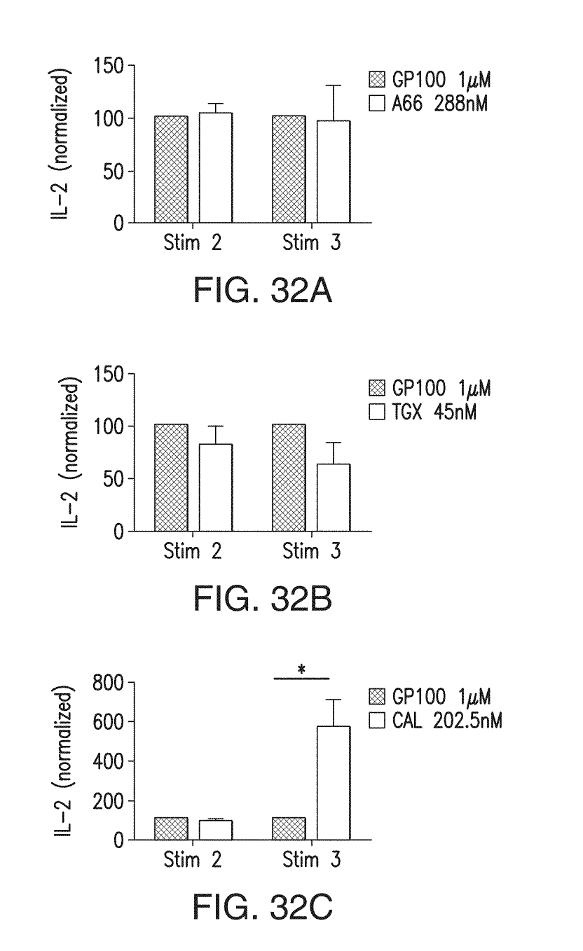

FIGS. 32A-32C show CAL-101 treated CD8 T-cells secrete significantly higher levels of IL-2 following stimulation 3, which is consistent with their higher proliferative potential. Data normalized to GP100, * p<0.05.

### Example 9: FIG. 5: PI3K-δ Controls the Differentiation of CD8 T-Cells Through the Downstream Akt1 and Akt2

FACS sorted CD8 T-cells from WT mice were activated in the presence or absence of A66 (288 nM), TGX-221 (45 nM) or CAL-101 (202.5 nM). The expression level of Akt1, Akt2 and Akt3 and the phosphorylation of these isoforms was assessed after three days of activation.

The phosphorylation of Akt1 and Akt2 was significantly reduced in the presence of PI3K-δ inhibitor, however the inhibition of PI3K-α or PI3K-β had no effect. Akt3 phosphorylation was not affected by the presence of any of the inhibitors (data not shown).

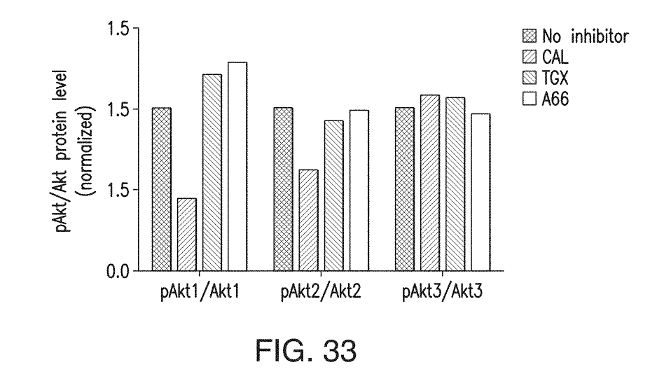

FIG. 33 shows densitometry of phosphorylated Akt isoform levels to the total Akt isoform ratio showed significantly lower levels of Akt1 and Akt 2 in the presence of PI3K-δ inhibitor. Densitometry was performed on the gel in A (not shown).

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

We claim:

1. A method for delaying or inhibiting terminal differentiation of CD8 T cells in a subject comprising administering to the subject an effective amount of an inhibitor of PI3K-δ.

2. A method for maintaining T_{CM} phenotype of T cells comprising contacting the T cells with an effective amount of an inhibitor of PI3K-δ to delay terminal differentiation and maintain the T_{CM} phenotype of the T cells.

3. A method for inhibiting or delaying exhaustion of CD8 T cells in a subject comprising administering to the subject an effective amount of an inhibitor of PI3K-δ to enhance the proliferative ability and survival of the CD8 T cells.

4. A method of modulating an immune response comprising administering an effective amount of an inhibitor of PI3K-δ to enhance the proliferative ability and survival of the CD8 T cells.

5. A method for reducing tumor burden in a subject in need there of comprising administering to the subject an effective amount of CD8 T cells treated with an inhibitor of PI3K-δ in combination or alternation with a tumor vaccine.

6. A method for treating cancer comprising administering to a subject in need thereof and effective amount of an inhibitor of PI3K-δ to delay or inhibit CD8 T cell exhaustion and enhance CD8 T cell proliferation and CD8 T cell cytokine production.

7. An immunotherapy composition comprising CD8 T cells treated with an effective amount of an inhibitor of PI3K-δ to delay or inhibit CD8 T cell exhaustion and enhance CD8 T cell proliferation and CD8 T cell cytokine production.

8. An immunotherapy regimen comprising administering to a subject in need thereof CD8 T cells treated with an effective amount of an inhibitor of PI3K-δ to delay or inhibit CD8 T cell exhaustion and enhance CD8 T cell proliferation and CD8 T cell cytokine production.
